(19) United States
(12) Patent Application Publication
(10) Pub. No.: US 2024/0266041 A1
(43) Pub. Date: Aug. 8, 2024

(54) MEDICAL INFORMATION PROCESSING APPARATUS, MEDICAL INFORMATION PROCESSING SYSTEM, AND MEDICAL INFORMATION PROCESSING METHOD

(71) Applicant: CANON MEDICAL SYSTEMS CORPORATION, Tochigi (JP)

(72) Inventors: Takahiko NISHIOKA, Otawara (JP); Toru TAKAHASHI, Nasushiobara (JP); Yasunari SHIOKAWA, Matsudo (JP); Masahiro OZAWA, Sakura (JP); Ko FUCHIGAMI, Ota-ku (JP); Miki YOSHIMURA, Nasushiobara (JP)

(73) Assignee: CANON MEDICAL SYSTEMS CORPORATION, Tochigi (JP)

(21) Appl. No.: 18/429,622

(22) Filed: Feb. 1, 2024

(30) Foreign Application Priority Data
Feb. 2, 2023 (JP) 2023-014848

(51) Int. Cl. G16H 40/63

(52) U.S. Cl. CPC G16H 40/63

(57) ABSTRACT

A medical information processing apparatus according to an embodiment includes processing circuitry configured: to acquire first information about a procedure performed on an examined subject so as to be kept in correspondence with time information; and to acquire, on the basis of the first information, second information about the procedure so as to be kept in correspondence with time information.

### CROSS-REFERENCE TO RELATED APPLICATIONS

This application is based upon and claims the benefit of priority from Japanese Patent Application No. 2023-014848, filed on Feb. 2, 2023; the entire contents of which are incorporated herein by reference.

### FIELD

Embodiments described herein relate generally to a medical information processing apparatus, a medical information processing system, and a medical information processing method.

### BACKGROUND

During a procedure performed on an examined subject or during a medical examination performed prior to a procedure, it is possible to acquire various types of information about the procedure. For example, it is possible to acquire examined subject information such as the height, the weight, and the like of the examined subject, measurement values such as blood pressure and a heart rate, a medical image taken of the examined subject, imaging conditions of the medical image, information indicating a medical tool used in the procedure, a video and/or audio recording situations inside the room while the procedure is performed. By acquiring and using the information about the procedure, it is expected that the procedure is made more efficient and that the precision level thereof is enhanced.

### BRIEF DESCRIPTION OF THE DRAWINGS

FIG. 1 is a block diagram illustrating an exemplary configuration of a medical information processing system according to a first embodiment;

FIG. 2 is a block diagram illustrating an exemplary configuration of an X-ray diagnosis apparatus according to the first embodiment;

FIG. 3 is a drawing illustrating an example of a display according to the first embodiment;

FIG. 4 is a block diagram illustrating an exemplary configuration of a medical information processing system according to a second embodiment;

FIG. 5A is a drawing illustrating an example of a time axis according to the second embodiment;

FIG. 5B is a drawing illustrating an example of a display according to the second embodiment;

FIG. 5C is a drawing illustrating another example of the display according to the second embodiment;

FIG. 5D is a drawing illustrating yet another example of the display according to the second embodiment;

FIG. 5E is a drawing illustrating yet another example of the display according to the second embodiment;

FIG. 5F is a drawing illustrating yet another example of the display according to the second embodiment;

FIG. 5G is a drawing illustrating yet another example of the display according to the second embodiment;

FIG. 6 is a block diagram illustrating an exemplary configuration of a medical information processing system according to a third embodiment;

FIG. 7A is a drawing illustrating an example of a time axis according to the third embodiment; and

FIG. 7B is a drawing illustrating an example of a display according to the third embodiment.

### DETAILED DESCRIPTION

A medical information processing apparatus according to an embodiment includes a first acquiring unit configured to acquire first information about a procedure performed on an examined subject so as to be kept in correspondence with time information; and a second acquiring unit configured to acquire, on the basis of the first information, second information about the procedure so as to be kept in correspondence with time information.

Exemplary embodiments of a medical information processing apparatus, a medical information processing system, and a medical information processing method will be explained in detail below, with reference to the accompanying drawings.

### First Embodiment

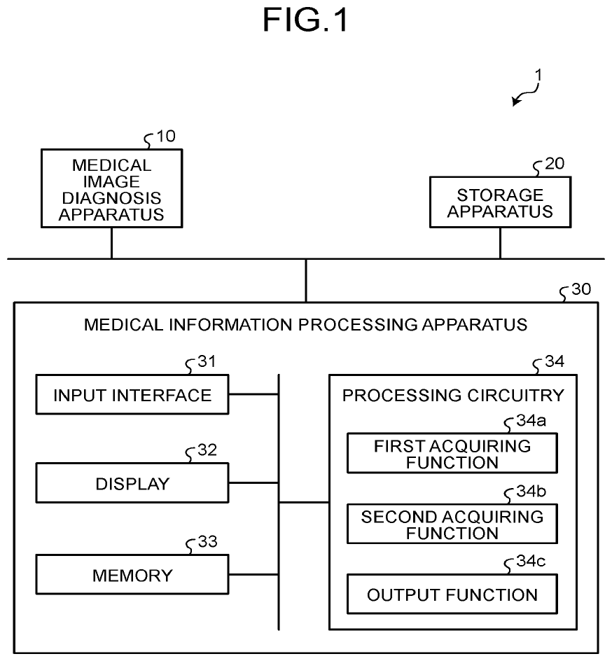

In a first embodiment, an example of a medical information processing system 1 including a medical information processing apparatus 30 will be explained. For example, as illustrated in FIG. 1, the medical information processing system 1 includes a medical image diagnosis apparatus 10, a storage apparatus 20, and the medical information processing apparatus 30. FIG. 1 is a block diagram illustrating an exemplary configuration of the medical information processing system 1 according to the first embodiment.

As illustrated in FIG. 1, the medical image diagnosis apparatus 10, the storage apparatus 20, and the medical information processing apparatus 30 are connected together via a network NW. In this situation, the network NW may be structured with a local network enclosed within a hospital or may be a network intermediated by the Internet. For example, the storage apparatus 20 may be installed in the same facility as the facility where the medical image diagnosis apparatus 10 and the medical information processing apparatus 30 are installed or may be installed in a different facility.

The medical image diagnosis apparatus 10 is configured to acquire a medical image to be used for a procedure performed on an examined subject (hereinafter, “patient”). For example, the medical image diagnosis apparatus 10 is configured to acquire the medical image of the patient before the procedure is started. By using the medical image, a user (e.g., a medical doctor) is able to explore a treatment plan including the state of a disease of the patient and whether or not the procedure is required. Further, for example, the medical image diagnosis apparatus 10 is configured to acquire a medical image of the patient while the procedure is performed. By using the medical image, the user is able to proceed with the procedure, while understanding an internal structure of the patient and the state of a tool inserted in the body of the patient. The medical images acquired by the medical image diagnosis apparatus 10 are examples of information about the procedure performed on a patient P.

The storage apparatus 20 is configured to store therein various types of information about the procedure performed on the patient P. For example, the storage apparatus 20 is a server of an information management system such as a Radiology Information System (RIS), a Hospital Information System (HIS), or a Picture Archiving and Communication System (PACS). Specific examples of the information stored by the storage apparatus 20 will be explained later.

The medical information processing apparatus 30 is configured to assist using the information about the procedure performed on the patient, through processes performed by processing circuitry 34 (explained later). For example, as illustrated in FIG. 1, the medical information processing apparatus 30 includes an input interface 31, a display 32, a memory 33, and the processing circuitry 34.

The input interface 31 is configured to receive various types of input operations from the user, to convert the received input operations into electrical signals, and to output the electrical signals to the processing circuitry 34. For example, the input interface 31 is realized by using a mouse, a keyboard, a trackball, a switch, a button, a joystick, a touchpad on which input operations can be performed by touching an operation surface thereof, a touch screen in which a display screen and a touchpad are integrally formed, contactless input circuitry using an optical sensor, audio input circuitry, and/or the like. Alternatively, the input interface 31 may be configured by using a tablet terminal or the like capable of wirelessly communicating with a main body of the medical information processing apparatus 30. Further, the input interface 31 may be circuitry configured to receive input operations from the user through a motion capture scheme. In an example, the input interface 31 is capable of receiving, as the input operations, body movements and lines of sight of the user as a result of processing a signal obtained via a tracker and/or an image acquired of the user. Furthermore, the input interface 31 does not necessarily need to include physical operation component parts such as the mouse, the keyboard, and/or the like. For instance, possible examples of the input interface 31 include electrical signal processing circuitry configured to receive an electrical signal corresponding to an input operation from an external input mechanism provided separately from the medical information processing apparatus 30 and to output the electrical signal to the processing circuitry 34.

The display 32 is configured to display various types of information. For example, under control of the processing circuitry 34, the display 32 is configured to display the information about the procedure performed on the patient P. Further, for example, the display 32 is configured to display a Graphical User Interface (GUI) used for receiving various types of instructions, settings, and the like from the user via the input interface 31. For example, the display 32 may be a liquid crystal display or a Cathode Ray Tube (CRT) display. The display 32 may be of a desktop type or may be configured by using a tablet terminal or the like capable of wirelessly communicating with the main body of the medical information processing apparatus 30.

Further, the display 32 may be provided in a medical examination room (hereinafter, “examination room”) where the procedure for the patient P is performed or may be provided in a room different from the examination room. For example, other than being provided in an examination room where an X-ray diagnosis apparatus 11 is installed, the display 32 may be provided in an operation room next to the examination room or in an image interpretation room. When the display 32 is provided in the examination room, it is possible to present a display for a user such as the medical doctor performing the procedure, a medical provider, or the like. In contrast, when the display 32 is provided in a room different from the examination room, it is possible to present a display for a user who monitors how the procedure is performed. As another example, the display 32 is also capable of presenting a display so that, after the procedure is finished, progress of the procedure can be checked retrospectively.

Although the example is explained with reference to FIG. 1 in which the medical information processing apparatus 30 includes the display 32, the medical information processing apparatus 30 may include a projector, in place of or in addition to the display 32. Under the control of the processing circuitry 34, the projector is capable of projecting an image on a screen, a wall, a floor, the body surface of the patient P, or the like. In an example, the projector is also capable of projecting an image on an arbitrary plane, object, space, or the like, by using a projection mapping scheme.

The memory 33 is realized, for example, by using a semiconductor memory element such as a Random Access Memory (RAM) or a flash memory, or a hard disk, an optical disk, or the like. For example, the memory 33 is configured to store therein a program used by circuitry included in the medical information processing apparatus 30 for realizing the functions thereof. In an example, the memory 33 may be realized by using a server group (a cloud) connected to the medical information processing apparatus 30 via the network NW.

The processing circuitry 34 is configured to control operations of the entirety of the medical information processing apparatus 30 by executing a first acquiring function 34a, a second acquiring function 34b, and an output function 34c. The first acquiring function 34a is an example of the first acquiring unit. The second acquiring function 34b is an example of the second acquiring unit. The output function 34c is an example of an output unit.

For example, the processing circuitry 34 is configured to acquire first information about the procedure performed on the patient so as to be kept in correspondence with time information, by reading and executing a program corresponding to the first acquiring function 34a from the memory 33. Also, the processing circuitry 34 is configured to acquire, on the basis of the first information, second information about the procedure so as to be kept in correspondence with time information, by reading and executing a program corresponding to the second acquiring function 34b from the memory 33. Further, the processing circuitry 34 is configured to control transmission and reception of data performed via the network NW and displays presented by the display 32, by reading and executing a program corresponding to the output function 34c from the memory 33. Details of processes performed by the first acquiring function 34a, the second acquiring function 34b, and the output function 34c will be explained later.

In the medical information processing apparatus 30 illustrated in FIG. 1, the processing functions are stored in the memory 33 in the form of the computer-executable programs. The processing circuitry 34 is a processor configured to realize the functions corresponding to the programs, by reading and executing the programs from the memory 33. In other words, the processing circuitry 34 that has read the programs has the functions corresponding to the read programs.

Further, although the example was explained with reference to FIG. 1 in which the single piece of processing circuitry (i.e., the processing circuitry 34) realizes the first acquiring function 34a, the second acquiring function 34b, and the output function 34c, it is also acceptable to structure the processing circuitry 34 by combining together a plurality of independent processors, so that the functions are realized as a result of the processors executing the programs. Further, the processing functions included in the processing circuitry 34 may be realized as being distributed among or integrated into one or more pieces of processing circuitry, as appropriate.

Furthermore, the processing circuitry 34 may be configured to realize the functions by using a processor of an external apparatus connected via the network NW. For example, the processing circuitry 34 may be configured to realize the functions illustrated in FIG. 1, by reading and executing the programs corresponding to the functions from the memory 33 and also using a server group (a cloud) connected to the medical information processing apparatus 30 via the network NW as computational resources.

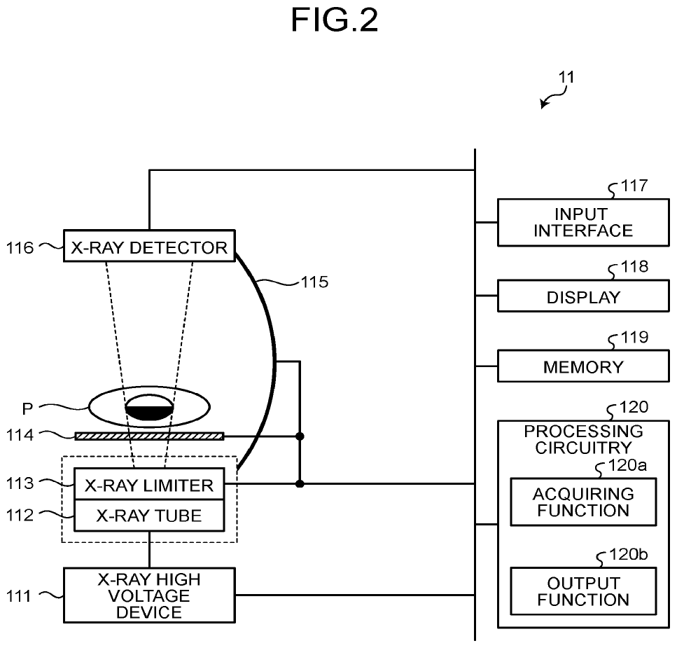

Next, as an example of the medical image diagnosis apparatus 10, the X-ray diagnosis apparatus 11 illustrated in FIG. 2 will be explained. FIG. 2 is a block diagram illustrating an exemplary configuration of the X-ray diagnosis apparatus 11 according to the first embodiment. As illustrated in FIG. 2, the X-ray diagnosis apparatus 11 includes an X-ray high-voltage device 111, an X-ray tube 112, an X-ray limiter 113, a tabletop 114, a C-arm 115, an X-ray detector 116, an input interface 117, a display 118, a memory 119, and processing circuitry 120.

Under control of the processing circuitry 120, the X-ray high-voltage device 111 is configured to supply high voltage to the X-ray tube 112. For example, the X-ray high-voltage device 111 includes: a high-voltage generating apparatus having electric circuitry such as a transformer and a rectifier and configured to generate the high voltage to be applied to the X-ray tube 112; and an X-ray controlling apparatus configured to control output voltage corresponding to X-rays to be emitted by the X-ray tube 112. The high-voltage generating apparatus may be of a transformer type or may be of an inverter type.

The X-ray tube 112 is a vacuum tube having a negative pole (a filament) that generates thermo electrons and a positive pole (a target) that generates the X-rays in response collisions of the thermo electrons therewith. By using the high voltage supplied from the X-ray high-voltage device 111, the X-ray tube 112 is configured to generate the X-rays by emitting the thermo electrons from the negative pole toward the positive pole.

The X-ray limiter 113 includes: a collimator configured to narrow down an emission range of the X-rays generated by the X-ray tube 112; and a filter configured to adjust the X-rays irradiated from the X-ray tube 112.

The collimator included in the X-ray limiter 113 includes four slidable limiting blades, for example. By sliding the limiting blades, the collimator is configured to narrow down the X-rays generated by the X-ray tube 112 so as to be emitted onto the patient P. In this situation, the limiting blades are plate-like members configured with lead or the like and are provided in the vicinity of an X-ray emission opening of the X-ray tube 112 for adjusting the emission range of the X-rays.

For the purpose of reducing a radiation exposure amount of the patient P and improving image quality of X-ray image data, the filter included in the X-ray limiter 113 is configured to reduce xeroradiographic components which are easily absorbed by the patient P and to reduce high-energy components which may bring about degradation of the contrast of the X-ray image data, by varying the quality of the X-rays passed thereby with the use of the material and/or the thickness thereof. Further, the filter is configured to attenuate the X-rays so that the X-rays emitted from the X-ray tube 112 onto the patient P has a predetermined distribution, by varying the dose and the emission range of the X-rays with the use of the material, the thickness, and/or the position thereof.

For example, the X-ray limiter 113 includes a driving mechanism such as a motor and an actuator or the like and is configured, under the control of the processing circuitry 120 (explained later), to control the X-ray emission by bringing the driving mechanism into operation. For example, the X-ray limiter 113 is configured to control the emission range of the X-rays emitted onto the patient P, by adjusting an opening degree of the limiting blades of the collimator by applying drive voltage to the driving mechanism in accordance with a control signal received from the processing circuitry 120. Further, for example, the X-ray limiter 113 is configured to control a dose distribution of the X-rays emitted onto the patient P, by adjusting the position of the filter by applying drive voltage to the driving mechanism in accordance with a control signal received from the processing circuitry 120.

The tabletop 114 is a bed on which the patient P is placed and is arranged over a table (not illustrated). The patient P is not included in the X-ray diagnosis apparatus 11. For example, the table includes a driving mechanism such as a motor and an actuator or the like and is configured to control moving/tilting of the tabletop 114 by bringing the driving mechanism into operation under the control of the processing circuitry 120 (explained later). For example, the table is configured to move and to tilt the tabletop 114, by applying drive voltage to the driving mechanism in accordance with a control signal received from the processing circuitry 120.

The C-arm 115 is configured to hold the X-ray tube 112 with the X-ray limiter 113 and the X-ray detector 116, so as to oppose each other, while the patient P is interposed therebetween. For example, the C-arm 115 includes a driving mechanism such as a motor and an actuator or the like and is configured, under the control of the processing circuitry 120 (explained later) to rotate and to move, as a result of bringing the driving mechanism into operation. For example, the C-arm 115 is configured to control emission positions and emission angles of the X-rays, by rotating/moving the X-ray tube 112 with the X-ray limiter 113 and the X-ray detector 116 relative to the patient P, by applying drive voltage to the driving mechanism in accordance with a control signal received from the processing circuitry 120. Although the example was explained with reference to FIG. 2 in which the X-ray diagnosis apparatus 11 is a single plane apparatus, possible embodiments are not limited to this example. The X-ray diagnosis apparatus 11 may be a bi-plane apparatus.

For example, the X-ray detector 116 is an X-ray Flat Panel Detector (FPD) including detecting elements arranged in a matrix formation. The X-ray detector 116 is configured to detect X-rays that were emitted from the X-ray tube 112 and have passed through the patient P and to output a detection signal corresponding to a detected X-ray amount to the processing circuitry 120. In this situation, the X-ray detector 116 may be a detector of an indirect conversion type including a grid, a scintillator array, and an optical sensor array or may be a detector of a direct conversion type including a semiconductor element configured to convert X-rays that have become incident thereto into electrical signals.

Because it is possible to realize the input interface 117, the display 118, and the memory 119, in the same manner as the input interface 31, the display 32, and the memory 33 described above, detailed explanations thereof will be omitted. Further, the input interface 117 is configured to receive various types of input operations from a user, to convert the received input operations into electrical signals, and to output the electrical signals to the processing circuitry 120. Further, for example, under the control of the processing circuitry 120, the display 118 is configured to display a GUI used for receiving instructions from the user and various types of information about the procedure. For example, the memory 119 is configured to store therein programs that correspond to various types of functions and are read and executed by the processing circuitry 120.

The processing circuitry 120 is configured to control operations of the entirety of the X-ray diagnosis apparatus 11, by executing an acquiring function 120a and an output function 120b.

For example, the processing circuitry 120 is configured to acquire X-ray images of the patient P, by reading and executing a program corresponding to the acquiring function 120a from the memory 119. For example, the acquiring function 120a is configured to sequentially acquire the X-ray images on the basis of the X-rays that have passed through the patient P, during the procedure performed on the patient P.

For example, the acquiring function 120a is configured to control the dose and the on/off of the X-rays emitted onto the patient P, by adjusting the voltage to be supplied to the X-ray tube 112 by controlling the X-ray high-voltage device 111. Further, the acquiring function 120a is configured to control the emission range of the X-rays emitted onto the patient P, by controlling the operations of the X-ray limiter 113 and adjusting the opening degree of the limiting blades included in the collimator. In addition, the acquiring function 120a is configured to control the dose distribution of the X-rays by controlling the operations of the X-ray limiter 113 and adjusting the position of the filter. Also, the acquiring function 120a is configured to control an image taking position and an image taking angle, by controlling the operations of the C-arm 115 and varying the position and the angle of the C-arm 115 relative to the patient P. The position and the angle of the C-arm 115 may be referred to as an arm position. Furthermore, the acquiring function 120a is configured to generate the X-ray images on the basis of the detection signal received from the X-ray detector 116 and to store the generated X-ray images into the memory 119.

Further, the processing circuitry 120 is configured to control data transmission and reception performed via the network NW and displays presented by the display 118, by reading and executing a program corresponding to the output function 120b from the memory 119. For example, the output function 120b is configured to transmit the X-ray images acquired by the acquiring function 120a to the medical information processing apparatus 30 via the network NW and to cause the display 118 to display the X-ray images.

In the X-ray diagnosis apparatus 11 illustrated in FIG. 12, the processing functions are stored in the memory 119, in the form of computer-executable programs. The processing circuitry 120 is a processor configured to realize the functions corresponding to the programs, by reading and executing the programs from the memory 119. In other words, the processing circuitry 120 that has read the programs has the functions corresponding to the read programs.

Further, although the example was explained with reference to FIG. 2 in which the single piece of processing circuitry (i.e., the processing circuitry 120) realizes the acquiring function 120a and the output function 120b, it is also acceptable to structure the processing circuitry 120 by combining together a plurality of independent processors, so that the functions are realized as a result of the processors executing the programs. Further, the processing functions included in the processing circuitry 120 may be realized as being distributed among or integrated into one or more pieces of processing circuitry, as appropriate.

Furthermore, the processing circuitry 120 may be configured to realize the functions by using a processor of an external apparatus connected via the network NW. For example, the processing circuitry 120 may be configured to realize the functions illustrated in FIG. 2, by reading and executing the programs corresponding to the functions from the memory 119 and also using a server group (a cloud) connected to the X-ray diagnosis apparatus 11 via the network NW as computational resources.

Further, the medical information processing system 1 may include, as the medical image diagnosis apparatus 10, a modality apparatus of a type different from the X-ray diagnosis apparatus 11. For example, the medical information processing system 1 may include one or more of the following, in place of or in addition to the X-ray diagnosis apparatus 11: an X-ray Computed Tomography (CT) apparatus, a Magnetic Resonance Imaging (MRI) apparatus, an ultrasound diagnosis apparatus, a Single Photon Emission Computed Tomography (SPECT) apparatus, and a Positron Emission computed Tomography (PET) apparatus.

The medical information processing system 1 including the medical image diagnosis apparatus 10, the storage apparatus 20, and the medical information processing apparatus 30 has thus been explained. Next, processes performed in the medical information processing system 1 will be explained.

To begin with, in clinical situations, various types of information about a procedure have conventionally been acquired and provided for users such as medical doctors. For example, the X-ray diagnosis apparatus 11 is capable of acquiring the X-ray images of the patient P, so that the display 118 displays the X-ray images. Further, for example, an electrocardiograph is capable of obtaining an electrocardiogram of the patient P and causing a display included in the electrocardiograph to display the electrocardiogram. Further, for example, a sphygmomanometer is capable of measuring blood pressure of the patient P and causing a display included in the sphygmomanometer to display the blood pressure of the patient P.

In relation to the above, in recent years, a system has been developed so as to integrally display a plurality of types of information acquired by mutually-different apparatuses such as the X-ray diagnosis apparatus 11, the electrocardiograph, and the sphygmomanometer. By using such a system, even when a plurality of types of information about a procedure are acquired by a plurality of apparatuses, the user is able to easily understand the plurality of types of information.

Further, acquiring pieces of information about a procedure so as to be kept in correspondence with time information also makes it possible to compare a plurality of types of information with each other. For example, by acquiring an electrocardiogram and blood pressure values so as to be kept in correspondence with time information, it is possible to display the electrocardiogram and the blood pressure values together in one graph having a time axis. In this situation, the time information is not particularly limited, as long as the information indicates points in time at which the pieces of information about the procedure were acquired. For example, the time information may be information indicating dates and/or times or may be information indicating an elapsed period of time since the procedure was started.

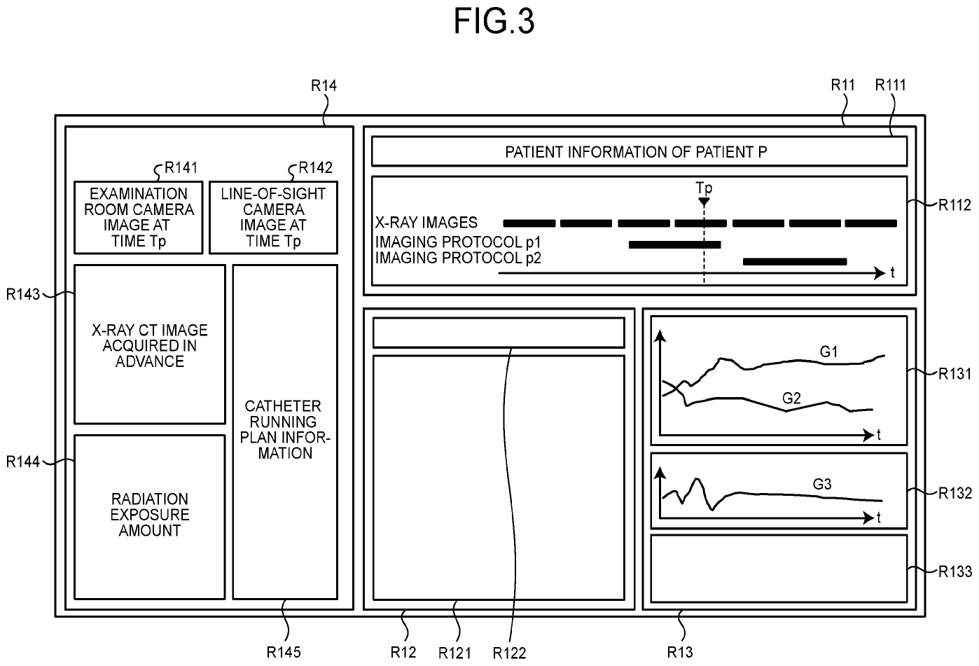

An example of a display integrating a plurality of types of information will be explained, with reference to FIG. 3. In FIG. 3, provided on a display screen are a display region R11, a display region R12, a display region R13, and a display region R14. As mentioned above, the display in FIG. 3 may be presented in the examination room or in another room. Further, the layout of the display region R11, the display region R12, the display region R13, and the display region R14 on the display screen is not limited to the example illustrated in FIG. 3 and may be substituted, changed, or deleted. For example, among the display regions displayed on the display screen, only the display region R11, the display region R12, and the display region R13 may be displayed, so that the display region R14 is brought into a non-display state or deleted in accordance with a user operation or a setting arranged in advance.

In the display region R11 in FIG. 3, the time series and events are displayed while being kept in correspondence with each other. More specifically, the display region R11 includes a display region R111 and a display region R112. Displayed in the display region R111 is patient information of the patient P such as a patient ID, his/her name, gender, and age, for example. Further, displayed in the display region R112 is a time axis t. On the time axis t, dates, times, and/or an elapsed period of time since the procedure was started may be displayed. On the time axis t, the time periods displayed with bars in the line corresponding to the “X-RAY IMAGES” are the time periods during which X-ray images were acquired. Further, on the time axis t, the time period displayed with a bar in the line corresponding to the “IMAGING PROTOCOL p1” is the time period during which X-ray images were acquired by using an imaging protocol p1. In addition, on the time axis t, the time period displayed with a bar in the line corresponding to the “IMAGING PROTOCOL p2” is the time period during which X-ray images were acquired by using an imaging protocol p2. Further, the time Tp in the display region R112 is a time selected by a user and may be a time of interest of the user, for example. The user is able to select the time Tp on the time axis t in the display region R112, for example, by performing a click operation using a mouse, a tap operation on a touch panel, or the like.

In the display region R12, examination information at the time Tp is displayed. More specifically, the display region R12 includes a display region R121 and a display region R122. Displayed in the display region R121 is an X-ray image taken at the time Tp, for example. Further, displayed in the display region R122 are imaging conditions of the X-ray image displayed in the display region R121. Examples of the imaging conditions of the X-ray image include an X-ray tube voltage value, an X-ray tube current value, an arm position, and a contrast enhancement condition.

Displayed in the display region R13 are a plurality of pieces of biological information data arranged in a time series. More specifically, the display region R13 includes a display region R131, a display region R132, and a display region R133. Displayed in the display region R131 are a graph G1 and a graph G2 kept in correspondence with the time axis t. Further, displayed in the display region R132 is a graph G3 kept in correspondence with the time axis t. The graph G1, the graph G2, and the graph G3 correspond to mutually-different pieces of biological information data. The time axis t in the display region R131 and the time axis t in the display region R132 may indicate mutually-different time spans. Further, possible methods for displaying the biological information data are not limited to graphs. For example, the biological information data may be displayed in the display region R133 by using text or a table.

Displayed in the display region R14 is relevant information about the examination at the time Tp. More specifically, the display region R14 includes a display region R141, a display region R142, a display region R143, a display region R144, and a display region R145. More specifically, displayed in the display region R141 is an examination room camera image at the time Tp. Displayed in the display region R142 is a line-of-sight camera image at the time Tp. The examination room camera image at the time Tp is an optical camera image taken at the time Tp by a camera installed on a wall surface or the ceiling of the examination room. The line-of-sight camera image at the time Tp is an optical camera image taken at the time Tp by a line-of-sight camera (a wearable camera) worn by the user such as a medical doctor or a medical provider. What is displayed in the display region R14 as the relevant information of the examination does not necessarily need to be of the procedure performed in a time period including the time Tp and may be information related to the patient P generated prior to the time period including the time Tp. For example, displayed in the display region R14 may be a procedure plan generated in advance prior to the procedure.

Further, displayed in the display region R143 is an X-ray CT image acquired in advance. For example, displayed in the display region R143 may be contrast-enhanced CT data acquired by using a contrast agent prior to the start of the procedure.

Further, displayed in the display region R144 is an accumulative radiation exposure amount of the patient P up to the time Tp. The radiation exposure amount displayed in the display region R144 does not necessarily need to be of the procedure performed in a time period including the time Tp and may be an accumulated radiation exposure amount from procedures performed on the patient P in the past. For example, displayed in the display region R144 may be a 3D model representing a human body in which colors corresponding to the radiation exposure amounts are displayed in different positions in the 3D model.

Further, displayed in the display region R145 is a catheter running plan information. For example, displayed in the display region R145 may be a blood vessel image including a treatment target site of the patient P. It is possible to acquire the blood vessel image by obtaining a difference between a contrast image acquired of the patient P having a contrast agent injected and a mask image acquired of the patient P while having no contrast agent injected. In this situation, the blood vessel image may be acquired by the X-ray diagnosis apparatus 11 or may be acquired by another apparatus (e.g., an X-ray CT apparatus or an MRI apparatus). Further, as the catheter running plan information, it is possible to display, in the blood vessel image, a path to reach the treatment target site.

As explained above, in recent years, using information about procedures is further promoted. In this regard, it is desirable when the amount and the types of the information about a procedure are enriched. Thus, the medical information processing apparatus 30 included in the medical information processing system 1 is configured to assist using the information about a procedure, by acquiring first information about the procedure and further acquiring second information about the procedure on the basis of the first information.

More specifically, to begin with, the first acquiring function 34a included in the processing circuitry 34a of the medical information processing apparatus 30 is configured to acquire the first information about a procedure performed on the patient P so as to be kept in correspondence with time information.

Examples of the first information include the medical images taken by the medical image diagnosis apparatus 10 such as the X-ray diagnosis apparatus 11. In that situation, the first acquiring function 34a is configured to acquire, from the medical image diagnosis apparatus 10, the medical images taken by the medical image diagnosis apparatus 10 so as to be kept in correspondence with the time information indicating points in time at which the medical images were taken. Alternatively, the first acquiring function 34a may acquire, via the network NW, medical images registered in a system such as a PACS and corresponding time information. In other words, the first acquiring function 34a may acquire the medical images and the corresponding time information from the storage apparatus 20. In this situation, the medical images acquired by the first acquiring function 34a may be still images or may be moving images. When the medical images are moving images, the time information is kept in correspondence with frames in the moving images.

Examples of the first information include imaging conditions of the medical images taken by the medical image diagnosis apparatus 10. For example, when the X-ray diagnosis apparatus 11 takes the X-ray images, the imaging conditions such as an X-ray tube voltage value, an X-ray tube current value, and an arm position are set. The first acquiring function 34a is configured to acquire, either directly from the medical image diagnosis apparatus 10 or via the storage apparatus 20, such imaging conditions of the medical images so as to be kept in correspondence with the time information indicating the points in time at which the medical images were taken. In this situation, when the medical image diagnosis apparatus 10 takes angiography images of the patient P, the imaging conditions include a contrast enhancement condition. The first acquiring function 34a may be configured to acquire the medical images to which the imaging conditions of the medical images are appended as accompaniment information.

Other examples of the first information include biological information data of the patient P, such as an electrocardiogram, a pulse rate, a heart rate, a respiration rate, blood pressure, a body temperature, a blood gas measurement value, and a Perfusion Index (PI). In this situation, the first acquiring function 34a is configured to acquire, via the network NW, the biological information data and corresponding time information from the apparatuses that measured the biological information data. In this situation, the blood gas measurement value is a measurement value related to the amount of gas contained in the blood or a pH value and, in an example, may be an arterial oxygen saturation level (SpO2).

The biological information data may be measured by individual apparatuses each corresponding to a different type of biological information data or may be measured by an apparatus (a polygraph) configured to simultaneously measure the plurality of types of biological information data. Further, the biological information data may be a record of numerical values or may be recorded as a waveform.

In another example, the first acquiring function 34a may be configured to acquire biological information data and corresponding time information by receiving an input operation from the user via the input interface 31. In yet another example, the first acquiring function 34a may be configured to acquire biological information data registered in a system such as an RIS or an HIS and corresponding time information via the network NW. In other words, the first acquiring function 34a may be configured to acquire the biological information data and the corresponding time information from the storage apparatus 20.

Other examples of the first information include information about a drug administered for the patient P. For example, the storage apparatus 20 may be configured to record therein, as the information about the drug, an administration history such as the type and the amount of the drug administered for the patient P as well as dates and times. In that situation, the first acquiring function 34a is capable of acquiring the information about the drug and the corresponding time information from the storage apparatus 20 via the network NW. In another example, the first acquiring function 34a is capable of acquiring the information about the drug and the corresponding time information by receiving an input operation from the user via the input interface 31.

Other examples of the first information include an optical camera image taken in the examination room where the procedure is performed. For example, for the purpose of recording situations with the doctor, medical providers, and the patient P while the procedure is performed on the patient P, a camera may be installed on a wall surface or the ceiling of the examination room. Further, while the procedure is performed, the medical doctor or a medical provider may wear a line-of-sight camera. The first acquiring function 34a is configured to acquire, either directly from the camera or via the storage apparatus 20, the optical camera images taken by such a camera, so as to be kept in correspondence with time information indicating the points in time at which the optical camera images were taken. The optical camera images acquired by the first acquiring function 34a may be still images or may be moving images. When the optical camera images are moving images, the time information is kept in correspondence with frames in the moving images.

In yet another example, the optical camera images may be images taken of the vicinity of the hands of the medical doctor. In yet another example, the optical camera images may be images taken of the vicinity of the hands of another medical doctor positioned next to the medical doctor. In other words, the optical camera images may be images taken of the vicinity of the hands of a first user or may be images taken of the vicinity of the hands of a second user different from the first user. In yet another example, the optical camera images may be images taken of the face of the patient P. From these optical camera images, it is possible to obtain information indicating reactions of the patient P such as facial expressions, lines of sight, eye movements, and/or the like, for instance. In yet another example, the optical camera images may be images taken of the face of a first user or images taken of the face of a second user. From these optical camera images, it is possible to obtain information indicating reactions of the user such as facial expressions, lines of sight, eye movements, and/or the like, for instance.

Other examples of the first information include audio recorded in the examination room where the procedure is performed. For example, for the purpose of recording speech of the medical doctor or a medical provider while the procedure is performed on the patient P, an audio recorder may be provided in the examination room. The first acquiring function 34a is configured to acquire, either directly from the audio recorder or via the storage apparatus 20, the audio recorded by the audio recorder, so as to be kept in correspondence with time information indicating the points in time at which the audio was recorded. Further, the first acquiring function 34a may be configured to integrally obtain the optical camera images and the audio, as the first information. In other words, the first acquiring function 34a may be configured to acquire moving images including the audio recorded in the examination room where the procedure is performed, as the first information.

The first information described above is merely an example. The first information may include any information recordable in relation to the procedure. For example, the first acquiring function 34a may be configured to acquire, as the first information, information about behaviors of the user such as a medical doctor and/or the patient P or a radiation exposure amount of the patient P caused by an X-ray image taking process or radiation treatment. It is possible to obtain the information about the behaviors, for example, by analyzing, facial expressions, lines of sight, eye movements, and the like, on the basis of optical camera images taken of the user and/or the patient P. Further, when the user is wearing a line-of-sight camera, it is possible to analyze the lines of sight of the user from the optical camera images taken by the line-of-sight camera.

The first acquiring function 34a is configured to acquire and store any of the various types of first information described above into the memory 33. In this situation, the output function 34c is capable of outputting the first information acquired by the first acquiring function 34a through the display 32.

As an example of the procedure performed on the patient P, an example will be explained in which intravascular intervention treatment is performed by using a catheter. In that situation, while the procedure is performed, the X-ray diagnosis apparatus 11 is configured to acquire angiography X-ray images in which the contrast of a blood vessel of the patient P is enhanced by using a contrast agent. Further, the first acquiring function 34a is configured to acquire the first information such as the X-ray images taken by the X-ray diagnosis apparatus 11 and the imaging conditions thereof, measurement results such as an electrocardiogram, the optical camera images taken in the examination room, the audio recorded in the examination room, and/or the like, so as to be kept in correspondence with the time information. After that, the output function 34c is configured to output a part or all of the first information acquired by the first acquiring function 34a through the display 32, so as to be kept in correspondence with one another, on the basis of the time information.

For example, while the procedure is performed, the output function 34c is configured to cause the display 32 to sequentially display the X-ray images being taken. In other words, the output function 34c is configured to display the X-ray images on the display 32 in a real-time manner. With this configuration, the user such as a medical doctor who is operating the catheter is able to proceed with the intravascular intervention treatment while understanding the structure of the blood vessel of the patient P and the position and the angle of the catheter inserted in the blood vessel.

Further, for example, the output function 34c is configured to display an electrocardiogram and blood pressure values together in one graph having a time axis. The graph is updated every time an electrocardiogram and a blood pressure value are newly measured. In other words, the output function 34c is configured to display, in the graph, transitions of the electrocardiogram and the blood pressure values within a predetermined time span including the present time.

Further, the output function 34c may be configured to output an X-ray image and imaging conditions thereof, an optical camera image, audio, and/or the like at a designated point in time in the past. For example, the user referencing the graph of the electrocardiogram and the blood pressure values is able to designate, via the input interface 31, a point in time of interest such as a point in time at which the blood pressure fell, for example. Accordingly, the output function 34c is capable of outputting an X-ray image taken at the designated point in time and the imaging condition thereof, an optical camera image and/or audio indicating the situation in the examination room at the designated point in time. With this configuration, it is possible to examine the reason why the blood pressure fell.

Next, processes performed by the second acquiring function 34b will be explained. On the basis of the first information described above, the second acquiring function 34b is configured to acquire the second information about the procedure performed on the patient P, so as to be kept in correspondence with time information.

For example, as the second information about the procedure, the second acquiring function 34b is configured to acquire information indicating a medical tool used in the procedure. For example, during intravascular intervention treatment, a guide wire is inserted up to the vicinity of a stenosis part of a blood vessel, so that a balloon catheter having a balloon at the tip end thereof is advanced along the guide wire, and the blood vessel is expanded by inflating the balloon catheter at the stenosis part, so as to place a stent in the expanded blood vessel. In this situation, when the X-ray images are acquired as the first information, the second acquiring function 34b is capable of acquiring the information indicating the medical tool used in the procedure, on the basis of the X-ray images.

More specifically, on the basis of the shape of the medical tool rendered in the X-ray images, the second acquiring function 34b is capable of recognizing which tool was operated at the points in time when the X-ray images were acquired, such as the “guide wire”, the “balloon catheter”, or the “stent”. In an example, as the second information kept in correspondence with the time information, the second acquiring function 34b is capable of acquiring events such as “time A1: started inserting the guide wire”, “time A2: started inserting the catheter”, “time A3: inflated the balloon”, and “time A4: placed the stent”.

Further, in some situations, the medical tool being used may be replaced, as appropriate. For example, after a catheter having a “diameter L1” is inserted in a blood vessel of the patient P, another catheter having a “diameter L2” which is smaller than the “diameter L1” may be inserted in the blood vessel in place of the catheter having the “diameter L1”, for the purpose of advancing the catheter into the blood vessel being narrower. The second acquiring function 34b may be configured to acquire information indicating that the medical tool has been replaced, so as to be kept in correspondence with time information. For example, as the second information kept in correspondence with the time information, the second acquiring function 34b is capable of acquiring an event such as “time A5: replaced the catheter having the diameter L1 with the catheter having the diameter L2”.

In this regard, when an analysis is performed only on the basis of the X-ray images, there may be a situation where it is impossible to identify the medical tool. For example, when a plurality of catheters having mutually-different model numbers are used, there may be a situation where, although it is possible to identify the insertion of each of the catheters on the basis of the X-ray images, it is impossible to identify the model number of the catheter being inserted.

To cope with this situation, the second acquiring function 34b may be configured to acquire the information indicating the medical tool being used, on the basis of a plurality of types of first information. For example, the second acquiring function 34b may be configured to acquire the information indicating the medical tool, on the basis of the optical camera images taken in the examination room where the procedure is performed and the X-ray images.

For example, when the catheter is rendered in an X-ray image, information about the catheter may appear in an optical camera image at the same time or at an earlier or later point in time. For example, when the catheter before being inserted in the blood vessel is rendered in an optical camera image, it is possible, in some situations, to identify the model number of the catheter on the basis of the shape and the color of the rendered catheter or text information or the like appended to the catheter. In another example, when a package of the catheter is rendered in an optical camera image, it is possible, in some situations, to identify the model number of the catheter, on the basis of text information or the like appended to the rendered package.

As explained above, the second acquiring function 34b is capable of enhancing a precision level and a reliability of the information indicating the medical tool, by using the plurality of types of first information at the time of acquiring the information indicating the medical tool. For example, although the example was explained in which the event “time A2: started inserting the catheter” is acquired on the basis of the X-ray images, the second acquiring function 34b is capable of identifying even the model number of the catheter used at the time A2, by further using the optical camera image in addition to the X-ray images.

Further, at the time of acquiring the information indicating the medical tool, the second acquiring function 34b may use registration information about medical tools that are usable in the facility. For example, the storage apparatus 20 may have registered therein the information about the medical tools that are managed in a usable state at the facility where the procedure for the patient P is performed. For example, the storage apparatus 20 may have registered therein information such as the model number, an ID, the manufacture year/month/day, dimensions such as the diameters and the length, the materials, and the like of each of the catheters that are usable at the facility. The second acquiring function 34b is capable of acquiring the information indicating the medical tool with a higher level of precision, by comparing analysis results based on the optical camera images and the X-ray images, with the registration information.

Further, although the example was explained in which the information indicating the medical tool was acquired by using the optical camera images and the X-ray images, it is possible to modify, as appropriate, the type of the first information being used. For example, when using a catheter, the medical doctor or a medical provider may utter the model number of the catheter. For example, when a medical provider hands the catheter to the medical doctor, the medical provider may utter the model number, an ID, or the like of the catheter, for the purpose of confirming that the handed catheter is the one designated in an instruction. In that situation, the second acquiring function 34b is capable of acquiring the information indicating the medical tool being used, on the basis of the audio and the X-ray images.

Further, specific methods for acquiring the information indicating the medical tool on the basis of the X-ray images, the optical camera images, the audio, and/or the like are not particularly limited. For example, the second acquiring function 34b may be configured to acquire the information indicating the medical tool, by using an arbitrary image recognition technique or audio recognition technique.

Alternatively, the second acquiring function 34b may acquire the information indicating the medical tool, by using a method based on machine learning. For example, the second acquiring function 34b may be configured to obtain and store into the memory 33, in advance, a trained model functioned to receive an input of an image and/or audio and to identify a medical tool. In this situation, the trained model may be generated by the second acquiring function 34b.

Alternatively, the trained model may be generated by another apparatus and obtained via the network NW. Further, when the first information such as the X-ray images has been acquired of the patient P, the second acquiring function 34b is able to acquire the information indicating the medical tool by inputting the first information to the trained model.

Other examples of the second information will be explained. At first, medical tools such as catheters are conventionally managed by using identifiers such as barcodes or identification numbers. When a medical tool is to be used, such an identifier is read. As a result, information indicating which medical tool was taken out at which point in time is recorded. The information of the read identifier is an example of the first information acquired by the first acquiring function 34a.

In this situation, the second acquiring function 34b may be configured to acquire information indicating the medical tool used in the procedure, on the basis of the optical camera images and the read information. More specifically, as explained above, it is possible to acquire the information indicating the medical tool on the basis of the optical camera images. Further, the information of the read identifier is a record indicating which medical tool was taken out at which point in time. By using these pieces of information in combination, the second acquiring function 34b is able to enhance the reliability of the information indicating the medical tool.

Further, there may be a situation where there is a discrepancy between the information indicating the medical tool and acquired on the basis of the optical camera images and the information of the read identifier. More specifically, there may be a situation where, although a medical tool was taken out by having the identifier read, the procedure is finished without using the medical tool due to a change in the state of the patient P or the like. In addition, there may be a situation where the procedure of having the identifier read may be forgotten at the time of taking out a medical tool. In those situations, the second acquiring function 34b is also capable of identifying the medical tool that was actually used, by acquiring the information indicating the medical tool on the basis of the optical camera images, and further correcting the information of the read identifier.

Further, although the example was explained in which the information indicating the medical tool is acquired by using the optical camera images and the read information, it is possible to change, as appropriate, the type of the first information being used. For example, the second acquiring function 34b is capable of acquiring the information indicating the medical tool being used, on the basis of the audio and the read information.

Other examples of the second information will be explained. As mentioned above, the first acquiring function 34a is configured to acquire, as the first information, the optical camera images taken in the examination room where the procedure is performed. In this situation, the second acquiring function 34b may be configured to acquire, as the second information, a clip image that is within the optical camera images and that includes a part rendering the medical tool used in the procedure.

More specifically, although it is possible to record, in the storage apparatus 20, the optical camera images acquired as the first information without applying any modification thereto, the optical camera images generally have a large data size and may impose a burden on the memory capacity of the storage apparatus 20 in some situations. In this regard, when the optical camera images are referenced later, what becomes of interest is often a partial region of the images such as the vicinity of the hands of the medical doctor operating the catheter inserted in the blood vessel, for example. Further, when a plurality of frames of optical camera images are acquired as moving images, what becomes of interest is often only one or more of the frames.

Accordingly, from the optical camera images acquired as the first information, the second acquiring function 34b may be configured to take out a partial region including a part rendering the medical tool or to take out a frame rendering the medical tool. In other words, at least in one of a spatial direction and a time direction, the second acquiring function 34b may take out a clip image including the part rendering the medical tool. With this configuration, it is possible to reduce the data size and to also enhance efficiency at the time of referencing the optical camera images later.

Other examples of the second information will be explained. As explained above, the second acquiring function 34b is capable of acquiring the information indicating the medical tool on the basis of the optical camera images. In this regard, when the medical tool is a tool used for administering a drug for the patient P, the second acquiring function 34b may be configured to acquire information indicating the type of the drug, as the second information. In other words, the first acquiring function 34a may be configured to acquire the optical camera images rendering the medical tool used for administering the drug for the patient P as the first information, whereas the second acquiring function 34b may be configured to acquire the information indicating the type of the drug on the basis of the optical camera images, as the second information.

For example, a three-way stopcock may be used for administering a drug for the patient P during a procedure. The three-way stopcock is a medical tool having a plurality of introduction ports. By having a plurality of types of drugs introduced through the introduction ports, it is possible to administer the drugs for the patient P while mixing the drugs together. In this situation, generally speaking, when such a three-way stopcock is used, the type of the drug to be introduced is determined for each of the introduction ports. Accordingly, the second acquiring function 34b is capable of estimating the type of the drug administered for the patient P, by identifying, in the optical camera images, one of the introduction ports of the three-way stopcock through which the drug is introduced. In one example, the second acquiring function 34b is capable of acquiring events such as “time A6: administered a drug B1” and “time A7: administered a drug B2”, as the second information kept in correspondence with time information.

Other examples of the second information will be explained. As explained above, the first acquiring function 34a is capable of acquiring, as the first information, the audio recorded in the examination room where the procedure is performed. The second acquiring function 34b may be configured to acquire the information indicating the type of the drug, on the basis of the audio. In other words, at the time of administering the drug, the user administering the drug may read the name of the drug to be administered for the patient P for a verification purpose, or an instruction including the name of the drug may be given to the user administering the drug. The second acquiring function 34b is capable of acquiring the information indicating the type of the drug, by analyzing the audio in that situation.

The second information acquired by the second acquiring function 34b is output by the output function 34c in the same manner as the first information described above. In this situation, the output function 34c may be configured to display the first information and the second information together.

For example, as explained above, the second acquiring function 34b is capable of acquiring, as the second information kept in correspondence with the time information, the events such as “time A1: started inserting the guide wire”, “time A2: started inserting the catheter”, “time A3: inflated the balloon”, “time A4: placed the stent”, “time A5: replaced the catheter having the diameter L1 with the catheter having the diameter L2”, “time A6: administered the drug B1”, and “time A7: administered the drug B2”. For example, the second acquiring function 34b is capable of causing the first information such as an electrocardiogram and blood pressure values to be displayed in one graph having a time axis and further causing the events identified as the second information to be displayed at the corresponding times in the graph.

As explained above, the medical information processing apparatus 30 according to the first embodiment includes the first acquiring function 34a and the second acquiring function 34b. The first acquiring function 34a is configured to acquire the first information about the procedure performed on the patient P so as to be kept in correspondence with the time information. On the basis of the first information, the second acquiring function 34b is configured to acquire the second information about the procedure so as to be kept in correspondence with the time information. With this configuration, the medical information processing apparatus 30 is able to assist using the information about the procedure performed on the patient. In other words, the medical information processing apparatus 30 is configured to acquire not only the first information, which is primary information acquired by the X-ray diagnosis apparatus 11 and other apparatuses such as an electrocardiograph, a sphygmomanometer, and/or a camera, but also the second information based on the first information. With this configuration, the medical information processing apparatus 30 is able to enrich the types and the amounts of the information about the procedure and to enhance the precision levels thereof.

### Second Embodiment

In a second embodiment, examples of the display will be explained. In the following sections, some of the elements that were explained in the first embodiment will be referred to by using the same reference characters, and duplicate explanations thereof will be omitted.

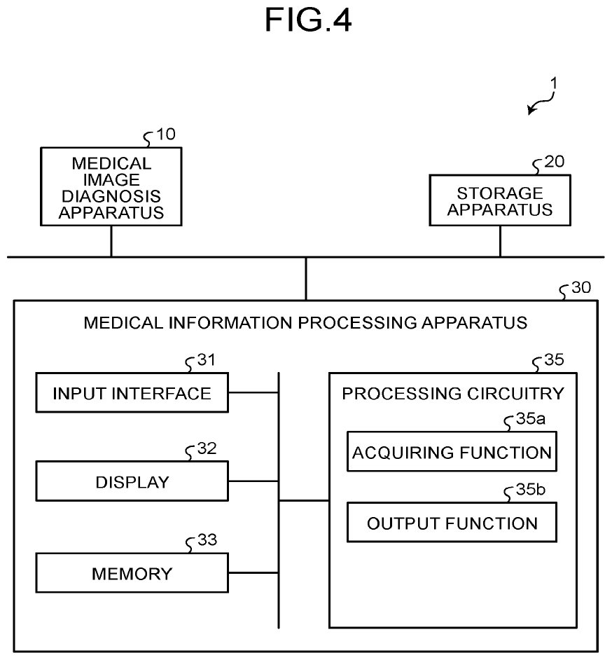

FIG. 4 illustrates an exemplary configuration of the medical information processing system 1 according to a second embodiment. The medical information processing system 1 according to the second embodiment includes, similarly to FIG. 1, the medical image diagnosis apparatus 10, the storage apparatus 20, and the medical information processing apparatus 30. Further, as illustrated in FIG. 4, the medical information processing apparatus 30 according to the second embodiment includes the input interface 31, the display 32, the memory 33, and processing circuitry 35.

The processing circuitry 35 is configured to control operations of the entirety of the medical information processing apparatus 30 by executing an acquiring function 35a and an output function 35b. The acquiring function 35a is an example of an acquiring unit. The output function 35b is an example of an output unit. For example, the processing circuitry 35 is configured to acquire information about a procedure performed on the patient P so as to be kept in correspondence with time information, by reading and executing a program corresponding to the acquiring function 35a from the memory 33. Further, the processing circuitry 35 is configured to output the information acquired by the acquiring function 35a in accordance with corresponding time information, by reading and executing a program corresponding to the output function 35b from the memory 33.

Further, the processing circuitry 34 illustrated in FIG. 1 and the processing circuitry 35 illustrated in FIG. 4 may 4 may be integrated together as appropriate. For example, the acquiring function 35a may include functions corresponding to the first acquiring function 34a and the second acquiring function 34b described above. In other words, the acquiring function 35a may be configured to acquire the first information about the procedure performed on the patient P so as to be kept in correspondence with the time information and to acquire, on the basis of the first information, the second information about the procedure so as to be kept in correspondence with the time information.

The output function 35b is configured to output information about the procedure acquired by the acquiring function 35a. For example, while intravascular intervention treatment is performed, the X-ray diagnosis apparatus 11 is configured to sequentially acquire X-ray images in which the contrast of a blood vessel of the patient P is enhanced by using a contrast agent. Further, the acquiring function 35a is configured to sequentially acquire newly-acquired X-ray images, either directly from the X-ray diagnosis apparatus 11 or via the storage apparatus 20. In addition, the output function 35b is configured to cause the display 32 to sequentially display the X-ray images newly acquired by the acquiring function 35a. In other words, the output function 35b is configured to display the X-ray images in a real-time manner, on the display 32.

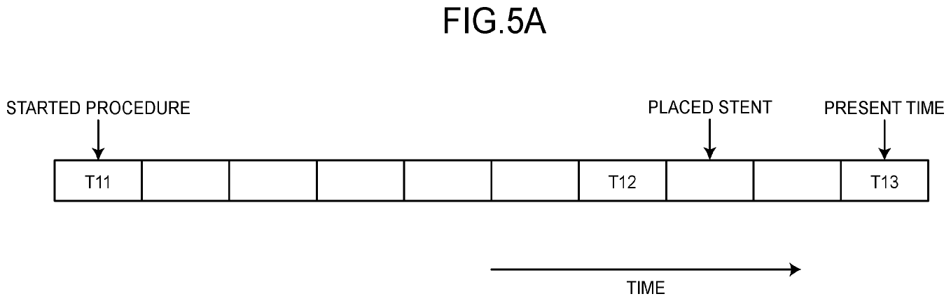

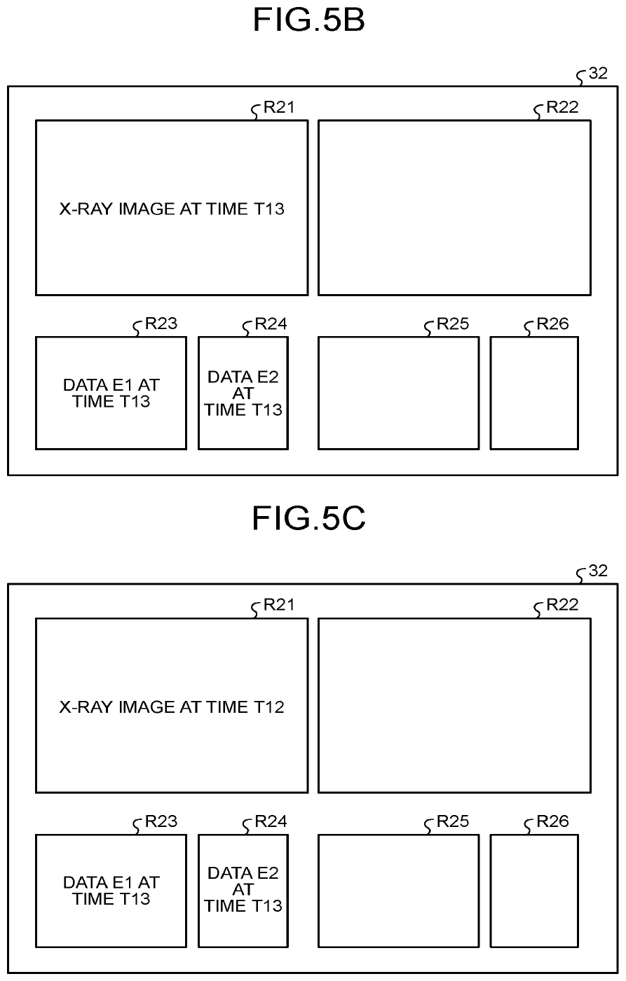

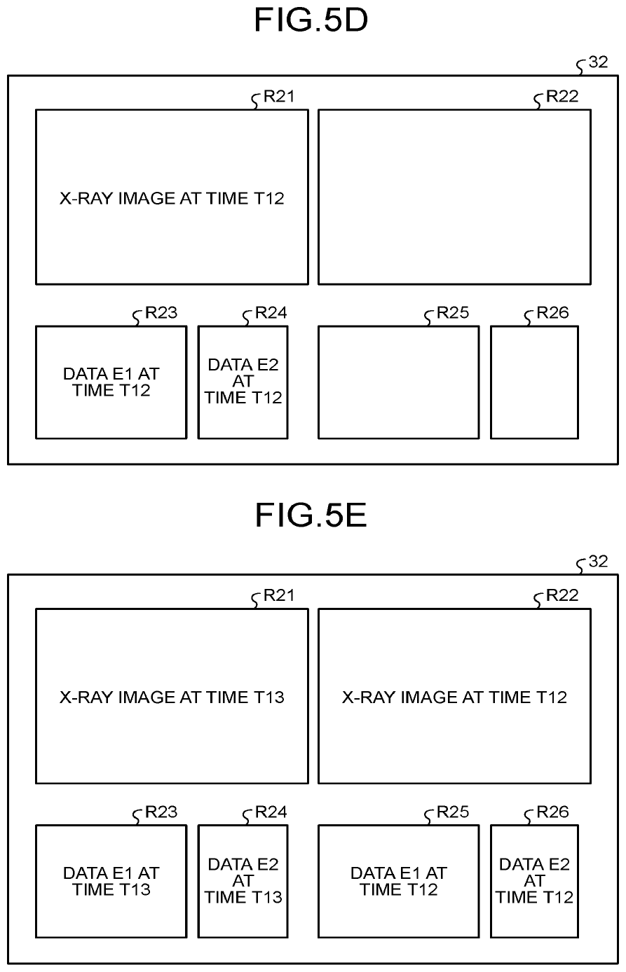

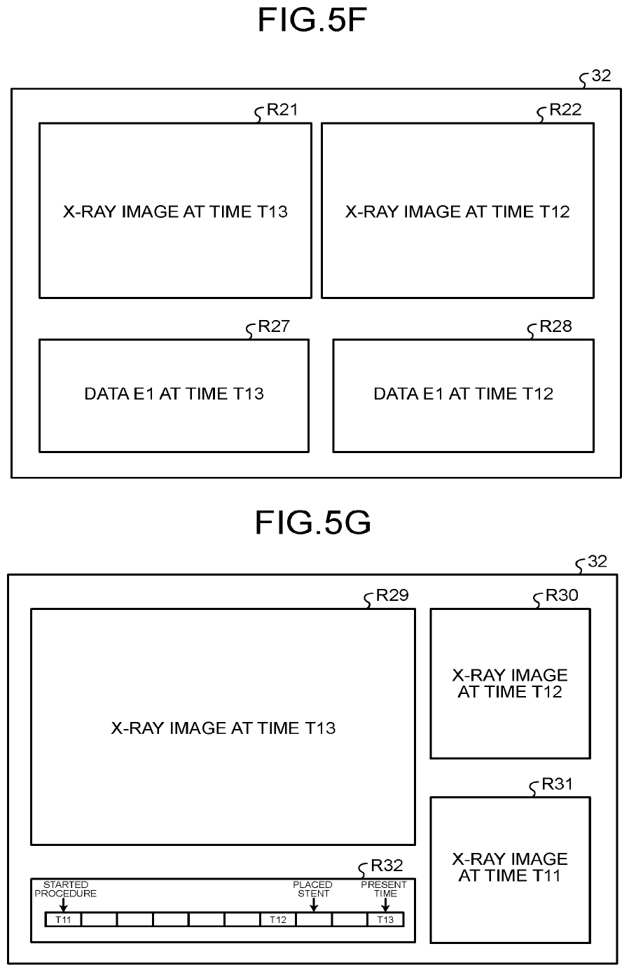

In this situation, the user such as a medical doctor may wish to reference information acquired in the past. An example will be explained in which, as illustrated in FIG. 5A, the point in time at which intravascular intervention treatment was started is expressed as time T11; a point in time before placement of a stent is expressed as time T12; and the present time is expressed as time T13.

For example, at time T13, the user may wish to cause an X-ray image from before the placement of the stent to be displayed, in order to check a change in the blood flow caused by the placement of the stent. In this situation, similarly to the second acquiring function 34b described above, the acquiring function 35a is capable of acquiring, as the second information, information indicating a medical tool used in the procedure so as to be kept in correspondence with time information. For example, as the second information kept in correspondence with the time information, the acquiring function 35a is capable of acquiring, in advance, an event such as “time A4: placed a stent”. In that situation, the output function 35b is capable of displaying an X-ray image acquired immediately before the time A4, as an X-ray image at time T12 which is a point in time before the placement of the stent.

Examples of displays presented by the output function 35b are illustrated in FIGS. 5B and 5C. In FIGS. 5B and 5C, provided on a display screen of the display 32 are a display region R21, a display region R22, a display region R23, a display region R24, a display region R25, and a display region R26.

Further, in FIG. 5B, displayed in the display region R21 is an X-ray image at time T13. In other words, in the display region R21, the output function 35b is displaying the X-ray image in a real-time manner. Together therewith, the output function 35b is displaying data E1 at time T13 in the display region R23 and displaying data E2 at time T13 in the display region R24. In other words, in the display region R23 and the display region R24, the output function 35b is displaying the data E1 and the data E2 of the patient P at present. In this situation, although the specific contents of the data E1 and the data E2 are not particularly limited, in an example, the output function 35b may display an electrocardiogram as the data E1 and display blood pressure as the data E2. The X-ray image is an example of a first type of information. The data E1 and the data E2 are examples of a second type of information.

In this situation, as illustrated in FIG. 5C, there may be a situation where, in order to check a change in the blood flow caused by the placement of the stent, for example, the image displayed in the display region R21 is changed to the “X-ray image at time T12” according to an instruction from the user. When the display has been changed in this manner, if the various types of data at time T13 were continuously displayed in the display region R23 and the display region R24 as illustrated in FIG. 5C, it will be difficult to understand the information because the mutually-different display regions will be displaying the information from the mutually-different points in time.

To cope with this situation, when the image displayed in the display region R21 has been changed to the “X-ray image at time T12”, the output function 35b is configured to also change the data displayed in the display region R23 and the display region R24 to data at time T12. More specifically, as illustrated in FIG. 5D, the output function 35b is configured to change what has been displayed in the display region R23 to “data E1 at time T12” and to change what has been displayed in the display region R24 to “data E2 at time T12”. With this configuration, the user is able to understand the various types of information at time T12 together and is also able to examine a relationship among those pieces of information.

As another example, when the user wishes to cause an X-ray image from before the placement of the stent to be displayed, the output function 35b may be configured, as illustrated in FIG. 5E, to additionally display the “X-ray image at time T12” in the display region R22, while keeping the “X-ray image at time T13” displayed in the display region R21. In that situation, the output function 35b is capable of displaying the various types of data at time T12 in the display region R25 and the display region R26, while keeping the various types of data at time T13 displayed in the display region R23 and the display region R24. More specifically, the output function 35b is capable of displaying the “data E1 at time T13” in the display region R23, displaying the “data E2 at time T13” in the display region R24, displaying the “data E1 at time T12” in the display region R25, and displaying the “data E2 at time T12” in the display region R26.

In the display example in FIG. 5E, the various types of data at time T13 are displayed in the display region R21, the display region R23, and the display region R24 arranged on the left side of the screen. In contrast, the various types of data at time T12 are displayed in the display region R22, the display region R25, and the display region R26 arranged on the right side of the screen. As explained herein, because the various types of data corresponding to each of the specific times are displayed somewhat collectively, it is possible to more easily understand with which time the pieces of data displayed in each of the display regions are kept in correspondence.

The display examples illustrated in FIGS. 5B to 5E are merely examples, and it is possible to apply various modifications thereto. For example, when the user wishes to cause the X-ray image from before the placement of the stent to be displayed, the output function 35b is configured, as illustrated in FIG. 5F, to additionally display the “X-ray image at time T12” in the display region R22, while keeping the “X-ray image at time T13” displayed in the display region R21. In addition, the output function 35b is configured to display the “data E1 at time T13” in a display region R27 and to display the “data E2 at time T13” in a display region R28. In other words, the examples were explained with reference to FIGS. 5B to 5E in which the two types of data (the data E1 and the data E2) are displayed as the second type of information; however, as illustrated in FIG. 5F, the data displayed as the second type of information may be one type of data.

FIG. 5G illustrates another example of the display. In FIG. 5G, the output function 35b is displaying the “X-ray image at time T13” in a display region R29, displaying the “X-ray image at time T12” in a display region R30, and displaying an “X-ray image at time T11” in a display region R31. In this situation, the display region R29 is a display region larger than the display region R30 and the display region R31. Further, in the display region R32, the output function 35b is displaying the time axis illustrated in FIG. 5A and the events corresponding to the various points in time.

For example, when time T13 is a time of interest for the user, the output function 35b is configured to display the “X-ray image at time T13” in the display region R29 and to also display the “X-ray image at time T12” and the “X-ray image at time T11” to be compared therewith in the display region R30 and the display region R31, which are smaller than the display region R29. Further, with the display in the display region R32, the output function 35b is also capable of indicating a chronological correspondence relationship among the X-ray images.

Needless to say, it is possible to change the X-ray image displayed in the display region R29, as appropriate. For example, when wishing to make an observation while using time T12 as a time of interest, the user selects “time T12” on the time axis in the display region R32, by performing a click operation using a mouse, a tap operation on a touch panel, or the like. At that time, the output function 35b is capable of changing the display in the display region R29 to the “X-ray image at time T12” and changing the display in the display region R30 to the “X-ray image at time T13”.

The “X-ray image at time T13” illustrated in FIGS. 5B to 5G is an example of first output information that is the first type of information and is kept in correspondence with time information indicating a first point in time. The “data E1 at time T13” and the “data E2 at time T13” are examples of second output information that is the second type of information different from the first type of information and is kept in correspondence with the time information indicating the first point in time. Further, the “X-ray image at time T12” is an example of third output information that is the first type of information and is kept in correspondence with time information indicating a second point in time different from the first point in time. Further, the “data E1 at time T12” and the “data E2 at time T13” are examples of fourth output information that is the second type of information and is kept in correspondence with the time information indicating the second point in time. At the time of displaying the third output information and the fourth output information, the third output information and the fourth output information may be displayed in place of the first output information and the second output information as illustrated in FIG. 5D or may be displayed in addition to the first output information and the second output information as illustrated in FIG. 5E.

The example was explained in which, as the X-ray image at time T12, the X-ray image acquired immediately before the placement of the stent is displayed; however, possible embodiments are not limited to this example. For instance, the output function 35b may be configured to identify the X-ray image at time T12 while taking imaging conditions into consideration. In other words, when the X-ray images at multiple points in time are displayed as illustrate in FIG. 5E or 5G, comparing the X-ray images with each other is easier when the imaging conditions thereof are more similar to each other. Thus, the output function 35b may display an X-ray image that can easily be compared with the X-ray image at time T13 as the X-ray image at time T12. On such occasion, the X-ray image at time T13 is an example of the first medical image kept in correspondence with the time information indicating the first point in time, whereas the X-ray image at time T12 is an example of the second medical image that is kept in correspondence with the time information indicating the second point in time and was acquired under an imaging condition similar to that of the first medical image.

For example, the output function 35b may be configured to display, as the X-ray image at time T12, an X-ray image having a similar arm position to that of the X-ray image at time T13, from among the X-ray images acquired before the time A4 at which the stent was placed. In other words, as the X-ray image at time T12, the output function 35b may be configured to display the X-ray image having a similar image taking angle and/or a similar image taking position to those of the real-time image.

Further, for example, the output function 35b may be configured to display, as the X-ray image at time T12, an X-ray image having a similar phase to that of the X-ray image at time T13, from among the X-ray images acquired before the time A4 at which the stent was placed. In other words, depending on image taking positions thereof, X-ray images may be affected, in some situations, by cyclical movements of the patient P such as heartbeats and/or respiration. In those situations, it is possible to make the comparison easier, by making the phases of the heartbeats and/or respiration match between the X-ray images being displayed. In that situation, it is possible to identify the phases of the heartbeats in the X-ray images by bringing the X-ray images into correspondence with an electrocardiogram on the basis of the time information. Further, it is possible to identify the phases of the respiration in the X-ray images by bringing the X-ray images into correspondence with a respiratory phase measurement result on the basis of the time information. It is possible to measure the respiratory phases by detecting the height of the surface of the chest of the patient P by using a depth sensor, for example. The respiratory phase measurement result is an example of the information about the procedure and is acquired by the acquiring function 35a.

Further, for example, the output function 35b is configured to display, as the X-ray image at time T12, an X-ray image having a similar contrast enhancement condition to that of the X-ray image at time T13, from among the X-ray images acquired before the time A4 at which the stent was placed. More specifically, in angiography X-ray images, the range of the blood vessel of which the contrast is enhanced, the contrast thereof, and the like vary depending on the amount or the position of the contrast agent injection, an elapsed period of time since the injection, and the like. The output function 35b is able to make the comparison easier, by displaying, as the X-ray image at time T12, the X-ray image having the similar contrast enhancement condition to that of the real-time image. The contrast enhancement condition is an example of the information about the procedure and is acquired by the acquiring function 35a.

As explained above, the medical information processing apparatus 30 according to the second embodiment includes the acquiring function 35a and the output function 35b. The acquiring function 35a is configured to acquire the information about the procedure performed on the patient P so as to be kept in correspondence with the time information. The output function 35b is configured to output the information about the procedure in accordance with the corresponding time information. With this configuration, the medical information processing apparatus 30 is able to assist using the information about the procedure performed on the patient.

Although the example was explained with reference to FIG. 5A in which the X-ray image at time T13 being a real-time image is displayed, while using time T13 as the present time, possible embodiments are not limited to this example. In other words, it is also acceptable to display a past image as the X-ray image at time T13. For example, when progress of the procedure is checked retrospectively after the procedure is finished, it is acceptable to display an X-ray image and/or an electrocardiogram at time T13 and/or time T12 being points in time in the past.

Further, as an example of the output of the output function 35b, the example using the display presented by the display 32 was explained; however possible embodiments are not limited to this example. For instance, FIGS. 5D and 5E depict the examples in which the data at time T12 is displayed. In that situation, in place of or in addition to the display of the data at time T12, it is also acceptable to output audio recorded at time T12. The output of the audio may be realized by the display 32 or may be realized by a speaker separate from the display 32.

### Third Embodiment

In a third embodiment, an example will be explained in which information about the procedure is analyzed so as to output a result thereof. In the following sections, some of the elements that were explained in the first and the second embodiments will be referred to by using the same reference characters, and duplicate explanations thereof will be omitted as appropriate.

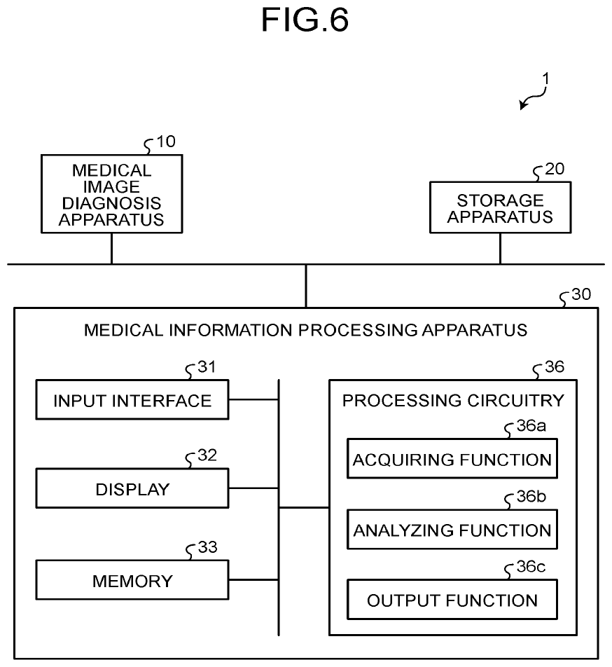

FIG. 6 illustrates an exemplary configuration of the medical information processing system 1 according to the third embodiment. Similarly to FIGS. 1 and 4, the medical information processing system 1 according to the third embodiment includes the medical image diagnosis apparatus 10, the storage apparatus 20, and the medical information processing apparatus 30. Further, as illustrated in FIG. 6, the medical information processing apparatus 30 according to the third embodiment includes the input interface 31, the display 32, the memory 33, and processing circuitry 36.

The processing circuitry 36 is configured to control operations of the entirety of the medical information processing apparatus 30 by executing an acquiring function 36a, an analyzing function 36b, and an output function 36c. The acquiring function 36a is an example of an acquiring unit. The analyzing function 36b is an example of an analyzing unit. The output function 36c is an example of an output unit. For example, the processing circuitry 36 is configured to acquire information about the procedure performed on the patient P so as to be kept in correspondence with time information, by reading and executing a program corresponding to the acquiring function 36a from the memory 33. Further, the processing circuitry 36 is configured to perform an analysis based on the information about the procedure, by reading and executing a program corresponding to the analyzing function 36b from the memory 33. Further, the processing circuitry 36 is configured to output a result of the analysis performed by the analyzing function 36b, by reading and executing a program corresponding to the output function 36c from the memory 33.

Further, the processing circuitry 34 illustrated in FIG. 1, the processing circuitry 35 illustrated in FIG. 4, and the processing circuitry 36 illustrated in FIG. 6 may be integrated together as appropriate. For example, the acquiring function 36a may include functions corresponding to the first acquiring function 34a and the second acquiring function 34b described above. In other words, the acquiring function 36a may be configured to acquire the first information about the procedure performed on the patient P so as to be kept in correspondence with the time information and to acquire, on the basis of the first information, the second information about the procedure so as to be kept in correspondence with the time information. Further, for example, the output function 36c may include a function corresponding to the output function 35b described above. In other words, the output function 36c may be configured to output the information about the procedure in accordance with the time information.

For example, as the information about the procedure, the acquiring function 36a is configured to acquire information indicating the type of a drug to be administered for the patient P. For example, similarly to the second acquiring function 34b described above, the acquiring function 36a is capable of acquiring the information indicating the type of the drug on the basis of an optical camera image rendering a medical tool used for administering the drug for the patient P. Alternatively, when the drug to be administered for the patient P is registered in a system such as an RIS or an HIS, the acquiring function 36a is capable of acquiring the information indicating the type of the drug from the system. In other words, the acquiring function 36a is capable of acquiring the information indicating the type of the drug from the storage apparatus 20.

The analyzing function 36b is configured to analyze suitability of the drug for the patient P, on the basis of the information indicating the type of the drug and patient information of the patient P. Examples of the patient information include a medical history and contraindication information. More specifically, the patient information includes information about a disease which the patient P is suffering from, records of the diseases which the patient P suffered from and the treatment which the patient P received in the past, and information about his/her constitution such as allergy. The analyzing function 36b may obtain the patient information from the storage apparatus 20 or may receive an input of the patient information via the input interface 31. For example, the patient information is registered into a system such as an HIS or an RIS, as a result of a consultation performed at the first visit of the patient P to the hospital.

In this situation, a relationship between the patient P and the drug to be administered for the patient P may correspond to a contraindication (not suitable). For example, it is known that there are some situations in which even a commonly-used drug may worsen symptoms or may cause nonnegligible side effects, when being administered for a patient having a specific disease. The analyzing function 36b is capable of judging whether the administration of the drug for the patient P corresponds to a contraindication, on the basis of the information about the type of the drug acquired by the acquiring function 36a and the patient information of the patient P.

The analysis performed by the analyzing function 36b may be performed in a rule-based manner or may be realized by using a method based on machine learning. For example, the analyzing function 36b is capable of analyzing the suitability for the patient P regarding the drug to be administered, by using a table defining correspondence to contraindications with respect to each combination of an item such as a disease, treatment, or constitution with a drug. As another example, the analyzing function 36b is also capable of analyzing the suitability for the patient P regarding the drug to be administered, by using a trained model functioned to receive inputs of an item such as a disease, treatment, or constitution and a drug and to further judge whether or not the combination corresponds to a contraindication.

The output function 36c is configured to output an analysis result of the analyzing function 36b. For example, when the drug to be administered for the patient P is determined to correspond to a contraindication as a result of the analysis performed by the analyzing function 36b, the output function 36c is configured to issue a notification indicating that the drug corresponds to a contraindication. In this situation, the notification may be issued via a display presented by the display 32 or may be issued via audio. Although the user such as a medical doctor does not usually decide to administer a drug corresponding to a contraindication, the notification issued by the output function 36c makes it possible to avoid, with higher certainty, the situation where a drug corresponding to a contraindication may be administered.

Other examples of the analysis performed by the analyzing function 36b will be explained. For instance, as the information about the procedure, the acquiring function 36a may be configured to acquire information indicating details of an instruction given by a first user performing the procedure to a second user and an optical camera image related to the second user.

The first user and the second user are each a medical doctor or a medical provider performing the procedure. For example, when multiple people perform the procedure for the patient P, an instruction about the procedure may be given from a primary doctor to another doctor besides himself/herself, a nurse, or other medical providers. In that situation, the first user is the primary doctor, whereas the second user is the medical provider receiving the instruction.

For example, the acquiring function 36a is configured to acquire audio recorded in the examination room, as the information indicating the details of the instruction given by the first user to the second user. As another example, the acquiring function 36a is configured to acquire an optical camera image taken by a line-of-sight camera worn by the second user, as an optical camera image related to the second user. As yet another example, the acquiring function 36a is configured to acquire, as the optical camera image related to the second user, an optical camera image being taken by a camera installed on a wall surface or the ceiling of the examination room and rendering the second user.

The analyzing function 36b is configured to analyze appropriateness of an action of the second user, on the basis of the information indicating the details of the instruction given by the first user to the second user and the optical camera image related to the second user. More specifically, the analyzing function 36b is configured to analyze whether or not the second user is acting according to the instruction from the first user.

As an example, a situation will be explained in which the first user instructs the second user to “administer a drug B3”. In this situation, the analyzing function 36b is capable of judging whether or not the drug is the “drug B3” from a label attached to a drug container held by the second user, for example, on the basis of the optical camera image. In another example, similarly to the second acquiring function 34b described above, the analyzing function 36b is capable of judging whether or not the drug to be administered by the second user for the patient P is the “drug B3” on the basis of an optical camera image rendering a medical tool used for administering the drug for the patient P.

As a different example, a situation will be explained in which the first user instructs the second user to “acquire an echo image from a position C1 in the esophagus at an angle C2” during an ultrasound image acquiring process using transesophageal echocardiography (TEE). In this situation, on the basis of an optical camera image rendering the vicinity of the hands of the second user operating an ultrasound probe, the analyzing function 36b is capable of judging whether or not the ultrasound probe is in the position and at the angle conforming to the instruction from the first user. In this situation, the analysis on the appropriateness of the action of the second user performed by the analyzing function 36b may be realized in a rule-based manner or may be realized by using a method based on machine learning.

The output function 36c is configured to output an analysis result of the analyzing function 36b. For example, when it is determined that the second user is not acting according to the instruction from the first user as a result of the analysis performed by the analyzing function 36b, the output function 36c is configured to notify the second user that his/her action is different from the instruction from the first user. For example, the output function 36c is capable of providing the second user with a comment such as “Please administer the drug B3” or “Please move the ultrasound probe to a position closer to the oral cavity”. Alternatively, the output function 36c may notify the first user that the second user is taking the action different from the instruction. The notification may be issued via a display presented by the display 32 or may be issued via audio. With the notification, it is possible to avoid, with higher certainty, misunderstanding the details of the instruction.

Other examples of the analysis performed by the analyzing function 36b will be explained. For example, as the information about the procedure, the acquiring function 36a may be configured to acquire one or more optical camera images related to the first user and the second user. In this situation, as explained above, the first user and the second user are each a medical doctor or a medical provider performing the procedure.

For example, as the optical camera images related to the first user and the second user, the acquiring function 36a is configured to acquire an optical camera image taken by a line-of-sight camera worn by the first user and an optical camera image taken by a line-of-sight camera worn by the second user. Alternatively, as the optical camera image related to the first user and the second user, the acquiring function 36a is configured to acquire an optical camera image being taken by a cameral installed on a wall surface or the ceiling of the examination room and rendering the first user and the second user.

The analyzing function 36b is configured to estimate a field of vision of each of the first and the second users, on the basis of the one or more optical camera images. For example, when the optical camera images are acquired by the line-of-sight cameras worn by the first user and the second user, the analyzing function 36b is capable of estimating a certain range including the center of each of the optical camera images as the field of vision of the corresponding one of the first and the second users. Further, when the optical camera image taken by the camera installed on the wall surface or the ceiling is acquired, by estimating a gaze point from the eye positions and the line-of-sight direction of each of the users, the analyzing function 36b is capable of estimating a certain range including the gaze point, as the field of vision of the corresponding one of the first and the second users.

Subsequently, the analyzing function 36b is configured to analyze information that is related to the patient P and is included only in the field of vision of one of the first and the second users. In the following sections, an example will be explained in which surgery is performed on the patient P. For example, while the first user is suturing a bleeding part of the patient P, there may be a situation where another bleeding part is present in a position that is not included in the field of vision of the first user, but is included in the field of vision of the second user. In that situation, there is a possibility that the bleeding part included only in the field of vision of the second user may be more serious than the bleeding part currently being sutured by the first user or that the bleeding part included only in the field of vision of the second user may not be recognized by the first user. In this situation, the bleeding part is an example of the information related to the patient P. The analysis on the information included only in the field of vision of one of the first and the second users may be realized in a rule-based manner or may be realized by using a method based on machine learning.

The output function 36c is configured to output information indicating that there is a bleeding part included only in the field of vision of the second user, as an analysis result of the analyzing function 36b. For example, the output function 36c is configured to notify the first user that there is a bleeding part included only in the field of vision of the second user. With this configuration, it is possible to address the bleeding parts more efficiently and to prevent the bleeding parts from being overlooked. Further, the output function 36c may be configured to cause the display 32 to display the optical camera image taken by the line-of-sight camera worn by the second user. With this configuration, users in the examination room are able to share the information about the bleeding parts.

Alternatively, the output function 36c may be configured to record, either in the memory 33 or the storage apparatus 20, the information indicating that there is a bleeding part included only in the field of vision of the second user, so as to be kept in correspondence with time information.

Other examples of the analysis performed by the analyzing function 36b will be explained. For instance, as the information about the procedure, the acquiring function 36a may be configured to acquire a medical image taken of a range including a medical tool inserted in a blood vessel of the patient P during intravascular intervention treatment and an optical camera image related to the user operating the medical tool.

For example, during the intravascular intervention treatment, the medical tool such as a guide wire, a catheter, or a stent is inserted in the blood vessel of the patient P and is operated by the user such as a medical doctor. In this situation, in order to understand the position and the angle of the medical tool inserted in the blood vessel, an X-ray image is acquired, as appropriate, by the X-ray diagnosis apparatus 11, for example. The acquiring function 36a is configured to acquire this X-ray image as the medical image taken of the range including the medical tool inserted in the blood vessel of the patient P. Further, as the optical camera image related to the user, the acquiring function 36a is configured to acquire an optical camera image taken by a line-of-sight camera worn by the user or an optical camera image taken by a camera installed on a wall surface or the ceiling of the examination room.

On the basis of the medical image and the optical camera image, the analyzing function 36b is configured to analyze appropriateness of the operation on the medical tool performed by the user. For example, it is possible to bend a part of the guide wire having a certain length from the tip end thereof, in accordance with an operation performed by the user. In that situation, quickly turning the guide wire while the tip end thereof is bent would impose a burden on the blood vessel of the patient P and is therefore not desirable. Thus, the analyzing function 36b is configured to estimate a degree of bending of the tip end part of the guide wire on the basis of the medical image and to estimate a rotation speed of the guide wire on the basis of the optical camera image. Thus, upon determining that a large burden is imposed on the blood vessel, the analyzing function 36b is configured to determine that the operation on the medical tool performed by the user is not appropriate. The analysis on the appropriateness of the operation on the medical tool may be realized in a rule-based manner or may be realized by using a method based on machine learning.

The output function 36c is configured to output an analysis result of the analyzing function 36b. For example, when it is determined that, during a guide wire operation, an operation imposing a large burden on the blood vessel is performed, the output function 36c is capable of notifying the user that the guide wire should be operated more carefully. The notification may be issued via a display presented by the display 32 or may be issued via audio. Alternatively, the output function 36c may be configured to record, either in the memory 33 or the storage apparatus 20, information indicating that an inappropriate operation was performed so as to be kept in correspondence with time information at which the operation was performed.

Other examples of the analysis performed by the analyzing function 36b will be explained. For example, as the information about the procedure, the acquiring function 36a may be configured to acquire a medical image taken of a range including a medical tool inserted in a blood vessel of the patient P during intravascular intervention treatment. The medical image may be an X-ray image acquired by the X-ray diagnosis apparatus 11, for example.

In this regard, there may be a situation in which, in addition to the medical tool such as a guide wire inserted in the body of the patient P for the purpose of the intravascular intervention treatment, another object may appear in the X-ray image. For example, there may be a situation in which a real-time X-ray image may render a medical tool that was placed in the body of the patient P in the past at a hospital different from the hospital where the current intravascular intervention treatment is being performed.

In the following sections, the medical tool inserted in the body of the patient P for the currently-performed intravascular intervention treatment will be referred to as a first medical tool. In contrast, the medical tool placed in the body of the patient P in a procedure different from the currently-performed intravascular intervention treatment will be referred to as a second medical tool. The second medical tool is an example of an object different from the first medical tool. The second medical tool may be related to a procedure performed at a different hospital or may have been placed in the body of the patient P for a long period of time. As a result, there may be a situation where the information about the second medical tool is not registered in a system such as an RIS or an HIS.

In this situation, the analyzing function 36b is configured to analyze the second medical tool on the basis of a medical image and specification information of the first medical tool. For example, when the first medical tool is a guide wire, the length of the bendable part at the tip end thereof is known specification information. The analyzing function 36b is capable of either obtaining the specification information of the guide wire registered in a system such as an RIS or an HIS or receiving an input of the specification information of the guide wire via the input interface 31.

Further, by using the length of the bendable part at the tip end of the guide wire as a reference, the analyzing function 36b is configured to analyze the second medical tool. For example, when the second medical tool is a stent that was placed in a blood vessel of the patient P in the past, the analyzing function 36b is capable of measuring dimensions such as the length and the width of the stent and coarseness of a strut or the like, by using the length of the bendable part at the time end of the guide wire as a reference. Further, on the basis of the measured dimensions, the analyzing function 36b is capable of estimating the type (e.g., the model number and/or the manufacturer) of the stent placed in the blood vessel of the patient P.

Alternatively, it is also possible to analyze the second medical tool only on the basis of the X-ray images. However, when using two-dimensional X-ray images, it is difficult to identify the position, in the depth direction, of the second medical tool being rendered. In addition, in X-ray images, the magnification ratio of the second medical tool varies depending on the position in the depth direction. For this reason, when analyzing the second medical tool, it is desirable to use information serving as a reference, such as the length of the bendable part at the tip end of the guide wire, for instance.

The second medical tool was explained as an example of the object different from the first medical tool; however, possible embodiments are not limited to this example. For instance, it is also possible to apply any of the above embodiments while using a blood vessel or a disease of the patient P as the object. In other words, the analyzing function 36b is capable of measuring the dimensions of the blood vessel or the disease of the patient P and estimating the type thereof, while using the length of the bendable part at the tip end of the guide wire, for example, as a reference. The analysis performed on the object by the analyzing function 36b may be performed in a rule-based manner or may be realized by using a method based on machine learning.

The output function 36c is configured to output the dimensions and/or the type of the object, as an analysis result of the analyzing function 36b. For example, when the object is a stent, the output function 36c is configured to notify the user of a measurement result such as the measured dimensions of the stent, the type of the stent, and/or the like. Alternatively, the output function 36c may record the dimensions and/or the type of the object into the memory 33 or the storage apparatus 20. For example, when the information about the second medical tool has not been registered in a system such as an HIS or an RIS, the output function 36c is configured to register therein the information about the second medical tool.

Further, for example, the output function 36c may be configured to update the registration information of the object, on the basis of the analysis result of the analyzing function 36b. For example, in some situations, the blood vessel of the patient P related to the procedure or a disease subject to the procedure may have been analyzed on the basis of an X-ray CT image or the like previously acquired, and the dimensions and/or the type thereof may have already been registered in a system such as an HIS or an RIS. In those situations, when the analyzing function 36b has performed the abovementioned analysis on the blood vessel or the disease, the output function 36c may update the registration information in the system such as the HIS or the RIS. Even when the previous analysis was appropriate, because the state of the blood vessel or the disease changes over the course of time, it is desirable to update the registration information thereof with the new information.

Other examples of the analysis performed by the analyzing function 36b will be explained. For instance, as the information about the procedure, the acquiring function 36a may be configured to acquire a medical image taken of a range including a medical tool inserted in a blood vessel of the patient P during intravascular intervention treatment. The medical image may be, for example, an X-ray image acquired by the X-ray diagnosis apparatus 11.

In this situation, there may be a situation where, as mentioned above, in addition to the first medical tool such as a guide wire inserted in the body of the patient P for the purpose of the intravascular intervention treatment, the X-ray image may render the second medical tool different from the first medical tool. As described above, the analyzing function 36b is configured to analyze the second medical tool on the basis of the medical image and the specification information of the first medical tool. For example, when the second medical tool is a stent that was placed in a blood vessel of the patient P in the past, the analyzing function 36b is configured to the measure dimensions such as the length and the width of the stent and coarseness of the strut or the like and to estimate the type of the stent, while using the length of the bendable part at the tip end of the guide wire as a reference.

Further, in accordance with the analysis result of the second medical tool, the analyzing function 36b is configured to select a third medical tool to be used in the intravascular intervention treatment. In other words, in accordance with the dimensions and/or the type of the second medical tool that was already embedded in the past treatment, the analyzing function 36b is configured to select the third medical tool to be used in the currently-performed intravascular intervention treatment.

In the following sections, an example will be explained in which, as a result of an analysis performed by the analyzing function 36b, the second medical tool is a stent of a type D1. Further, in the example explained below, the currently-performed intravascular intervention treatment is a procedure to place the stent in a stenosis part of a blood vessel of the patient P. In this situation, there may be candidates for the type of stent to be placed in the stenosis part in the currently-performed intravascular intervention treatment. In the following sections, an example will be explained in which the candidates for the stent to be used in the currently-performed intravascular intervention treatment include a stent of a type D2 and a stent of a type D3. In that situation, the analyzing function 36b is configured to judge which is more suitable between the stent of the type D2 and the stent of the type D3, as the third medical tool to be used together with the stent of the type D1 that is already embedded.

For example, in some situations, a system such as an HIS or an RIS may have recorded therein, as clinical results, prognoses of patients who had the stent of the type D1 and the stent of the type D2 embedded and patients who had the stent of the type D1 and the stent of the type D3 embedded. On the basis of the clinical results, the analyzing function 36b is capable of estimating, with respect to the patient P who already have the stent of the type D1 embedded, which will bring out a better prognosis between using the stent of the type D2 and using the stent of the type D3 and are thus capable of selecting the stent to be used. The selection of the third medical tool determined by the analyzing function 36b may be realized in a rule-based manner or may be realized by using a method based on machine learning.

The output function 36c is configured to output an analysis result of the analyzing function 36b. For example, the output function 36c is configured to notify the user of the third medical tool selected by the analyzing function 36b. For example, as described above, when the third medical tool has been selected on the basis of the prognoses of the patients recorded as the clinical results, the output function 36c may be configured to further notify the user of to what extent the prognosis of the patient P is expected to be improved by using the third medical tool selected by the analyzing function 36b. The user is able to determine the third medical tool to be used, while using the analysis result of the analyzing function 36b as reference information.

Other examples of the analysis performed by the analyzing function 36b will be explained. For instance, the acquiring function 36a may be configured to sequentially acquire medical images in which the contrast of a blood vessel of the patient P is enhanced, as the information about the procedure. For example, the medical images are X-ray images taken by the X-ray diagnosis apparatus 11 during intravascular intervention treatment. In the following sections, an X-ray image that is one of the medical images in which the contrast of the blood vessel is enhanced and that was acquired most recently by the acquiring function 36a may be referred to as a blood vessel fluoroscopic image. The blood vessel fluoroscopic image is a real-time image indicating a blood flow state of the patient at the present point in time or immediately prior.

The analyzing function 36b is configured to analyze the blood flow state in the blood vessel fluoroscopic image in accordance with a purpose of the procedure. For example, the analyzing function 36b is configured to compare the blood vessel fluoroscopic image with a past image, which is an angiography image acquired in the past. Examples of the past image include an X-ray CT image or an MRI image acquired before the procedure was started and an X-ray image acquired by the X-ray diagnosis apparatus 11 at a past point in time during the procedure.

There may be a situation where, when the blood vessel fluoroscopic image is compared with the past image, the quantity and/or the thicknesses of the blood vessels may have changed. For example, when the procedure performed on the patient P is to expand a stenosis part of a blood vessel, in some situations, because the contrast agent starts flowing to a blood vessel on the downstream side of the stenosis part as a result of the procedure, the quantity and/or the thicknesses of the blood vessels may increase. As another example, when the procedure to expand a stenosis part is performed, in some situations, because plaque in the vicinity of the stenosis part may break off to flow downstream and clog the blood flow, the quantity and/or the thicknesses of the blood vessels may decrease. In yet another example, when the state of a stenosis part became worse between the time when the past image was taken and the time when the fluoroscopic image is taken, the quantity and/or the thicknesses of the blood vessels may decrease in some situations.

As explained above, even when the quantity and/or the thickness of the blood vessels have changed between the blood vessel fluoroscopic image and the past image as explained above, there are some changes fulfilling the purpose of the procedure and other changes that are unintended. When the quantity and/or the thicknesses of the blood vessels have changed between the blood vessel fluoroscopic image and the past image, while the change does not match the purpose of the procedure, the analyzing function 36b is configured to identify the change as an unintended change in the blood flow state. The process of identifying the unintended change in the blood flow state performed by the analyzing function 36b may be realized in a rule-based manner or may be realized by using a method based on machine learning.

The output function 36c is configured to output the identified unintended change in the blood flow state, as an analysis result of the analyzing function 36b. For example, the output function 36c may be configured to notify the user of the unintended change in the blood flow state identified by the analyzing function 36b or configured to store the change in the memory 33 or the storage apparatus 20.

Although the example was explained in which the blood flow state is compared with that in the past image, what can be compared is not limited to the past image. For example, at the time of making a treatment plan on the basis of the past image such as an X-ray CT image, a blood flow state expected to be achieved by successful treatment is estimated. The analyzing function 36b may be configured to identify the unintended change in the blood flow state by comparing the blood flow state in the blood vessel fluoroscopic image with the blood flow state expected to be achieved by the successful treatment.

Further, for example, the analyzing function 36b may be configured to identify the unintended change in the blood flow state, on the basis of an electrocardiogram. For example, when the procedure performed on the patient P is to expand a stenosis part of a blood vessel, there is a possibility that an abnormal waveform may be detected from the electrocardiogram as a result of an increase in a blood flow volume. Thus, the analyzing function 36b may be configured to identify the blood flow state in a blood vessel fluoroscopic image at the time of the detection of the abnormal waveform in the electrocardiogram, as the unintended change in the blood flow state.

Other examples of the analysis performed by the analyzing function 36b will be explained. For example, as the information about the procedure, the acquiring function 36a may be configured to acquire time-series information. The time-series information is information successively acquired in the time direction, such as an electrocardiogram or blood pressure values, for example.

The analyzing function 36b may be configured to detect a fluctuation in the time-series information. For example, the analyzing function 36b is configured to detect, as the fluctuation, an abnormal waveform in the electrocardiogram or a drastic increase or decrease in the heart rate. As another example, the analyzing function 36b may be configured to detect, as the fluctuation, a drastic increase or decrease in the blood pressure values.

Further, the analyzing function 36b is configured to analyze the detected fluctuation, in accordance with information about a procedure performed for the patient P in relation to the procedure. For example, when the blood pressure drastically fell during a time period in which no particular procedure is performed for the patient P, the analyzing function 36b is configured to identify the drastic fall in the blood pressure as an unintended fluctuation in the time-series information.

In contrast, there may be a situation where, immediately before the blood pressure drastically fell, a procedure such as administration of a drug had been performed. In addition, there are some drugs that are administered to lower blood pressure, for the purpose of reducing burdens on the heart, for example. Thus, when the blood pressure fell as a result of a procedure performed for the patient P in this manner, the analyzing function 36b is configured to determine that the fluctuation is not an unintended fluctuation. Further, even when a fluctuation occurs immediately after a procedure such as administration of a drug or the like, the analyzing function 36b may determine the fluctuation to be an unintended fluctuation, depending on specifics of the procedure or the fluctuation. For example, when the blood pressure increases although a drug for lowering blood pressure was administered or when the blood pressure falls to a level significantly lower than a goal level, the analyzing function 36b may identify the fluctuation as an unintended fluctuation. The process of identifying the unintended fluctuation in the time-series information performed by the analyzing function 36b may be realized in a rule-based manner or may be realized by using a method based on machine learning.

The output function 36c is configured to output the identified unintended fluctuation in the time-series information, an analysis result of the analyzing function 36b. For example, the output function 36c may be configured to notify the user of the unintended fluctuation in the time-series information identified by the analyzing function 36b or configured to store the unintended fluctuation into the memory 33 or the storage apparatus 20.

The information about the procedure performed for the patient P is an example of the information about the procedure. For example, the information about the procedure is registered into a system such as an HIS or an RIS on the basis of an input operation performed by the user. In another example, the acquiring function 36a may acquire the information about the procedure performed for the patient P, on the basis of an optical camera image taken in the examination room.

Further, although the administration of the drug was explained as an example of the procedure performed for the patient P, possible embodiments are not limited to this example. Other examples of the procedure performed for the patient P include an operation performed on a medical tool inserted in a blood vessel, injection of a contrast agent, and surgical procedures such as incision and puncture.

Other examples of the analysis performed by the analyzing function 36b will be explained. For instance, as the information about the procedure, the acquiring function 36a may be configured to acquire information related to the patient P corresponding to a time after the procedure is started. Examples of such information include biological information data such as an electrocardiogram or blood pressure and a procedure performed for the patient P corresponding to a time after the procedure is started.

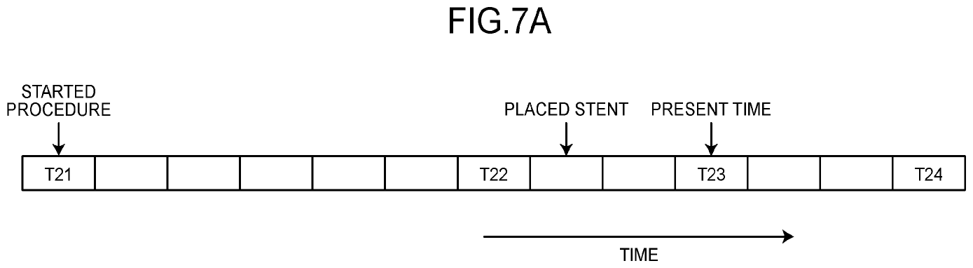

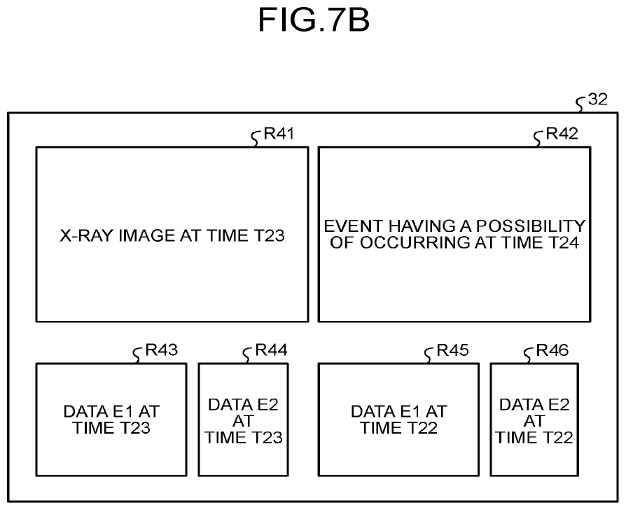

On the basis of the information related to the patient P corresponding to the time after the procedure is started, the analyzing function 36b may be configured to estimate a state change that may occur in the patient P. For example, as illustrated in FIG. 7A, a point in time at which intravascular intervention treatment was started is expressed as time T21, a point in time prior to placement of a stent is expressed as time T22, and the present time is expressed as time T23. In this situation, the analyzing function 36b is configured to estimate an event having a possibility of occurring at time T24, which is later than time T23. For example, the analyzing function 36b may estimate the possibility and a time of occurrence of ventricular fibrillation (VF).

For example, by using a method based on machine learning, the analyzing function 36b is configured to estimate the possibility and the time of the occurrence of ventricular fibrillation. For example, the analyzing function 36b is capable of generating a trained model functioned to estimate the possibility and the time of the occurrence of ventricular fibrillation, through a training in which changes in an electrocardiogram and blood pressure observed after the start of the procedure as well as patterns of procedures performed and the like are used as input-side data, while whether ventricular fibrillation occurred or not and the time at which ventricular fibrillation occurred are used as output-side data. In this situation, it is possible to acquire training data used for generating the trained model, for example, from clinical results of one or more patients who were subject to the same or a similar procedure as the patient P. By inputting the information related to the patient P to the trained model, the analyzing function 36b is capable of estimating the possibility and the time of the occurrence of ventricular fibrillation. Alternatively, the trained model may be generated by an apparatus different from the medical information processing apparatus 30. Further, the estimation made by the analyzing function 36b may be realized in a rule-based manner.

The output function 36c is configured to output an estimation result of the state change that may occur in the patient P, as an analysis result of the analyzing function 36b. As an example, an example of a display presented by the output function 36c is illustrated in FIG. 7B. In FIG. 7B, provided on a display screen of the display 32 are a display region R41, a display region R42, a display region R43, a display region R44, a display region R45, and a display region R46.

In FIG. 7B, displayed in the display region R41, the display region R43, and the display region R44 are an X-ray image at time T23, and the data E1 and the data E2 at time T23. In other words, displayed in the display region R41, the display region R43, and the display region R44 are information related to the patient P at present. In contrast, displayed in the display region R45 and the display region R46 are the data E1 and the data E2 at time T22. In other words, displayed in the display region R45 and the display region R46 are information related to the patient P from the past. Further, displayed in the display region R42 is an event having a possibility of occurring at time T24. For example, displayed in the display region R42 is an estimation result such as “The possibility of occurrence of ventricular fibrillation at time T24: High”. In other words, displayed in the display region R42 is information related to the patient P in the future. As explained herein, in the example in FIG. 7B, the output function 36c is capable of integrally displaying the information at the different point in time.

As explained above, the medical information processing apparatus 30 according to the third embodiment includes the acquiring function 36a, the analyzing function 36b, and the output function 36c. The acquiring function 36a is configured to acquire the information about the procedure performed on the patient P so as to be kept in correspondence with the time information. The analyzing function 36b is configured to perform the analysis based on the information about the procedure. The output function 36c is configured to output the analysis result of the analyzing function 36b. With this configuration, the medical information processing apparatus 30 is able to assist using the information about the procedure performed on the patient P. For example, the medical information processing apparatus 30 is able to further provide the information that might be difficult to understand if the information about the procedure were simply referenced and to enrich the information that will be kept as records.

In the first to the third embodiments, the examples of the intravascular intervention treatment using the stent were primarily explained; however, possible embodiments are not limited to those examples. It is possible to similarly apply the present embodiments to other various procedures performed while an X-ray image is referenced.

For example, it is possible to similarly apply any of the embodiments described above to procedures using: an electrode of an electrophysiological catheter used for treating arrhythmia; a drill of a Rotablator used for treating a firm stenosis part that is difficult to expand with a balloon or a stent; a metallic cylinder having a hole and being attached to the tip end of a catheter to be used for directional coronary resection; a catheter having a function of transmitting and receiving an ultrasound wave for examining a status in a blood vessel in a stenosis part; an intravascular endoscope; vascular ultrasound; intravascular Magnetic Resonance Imaging (MRI); Optical Coherence Tomography (OCT); a tool for transplanting stem cells in the field of regenerative medicine; an artificial valve; a vascular graft; and the like. In addition, while intravascular intervention treatment is a procedure performed while a medical tool is inserted in a blood vessel, it is possible to similarly apply any of the abovementioned embodiments to various types of procedures performed by inserting a medical tool into the body. For example, it is possible to similarly apply any of the above embodiments to laparoscopy. Furthermore, it is also possible to similarly apply any of the above embodiments to hybrid treatment of surgery and internal medicine and to a procedure such as guiding a puncture needle for biopsy in surgical treatment.

The term “processor” used in the above explanation denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). When the processor is a CPU, for example, one or more processors are configured to realize the functions by reading and executing the programs saved in the storage circuitry. In contrast, when the processor is an ASIC, for example, instead of having the programs saved in the storage circuitry, the functions are directly incorporated as logic circuitry in the circuitry of one or more processors. Further, the processors in the present embodiment do not each necessarily have to be structured as a single piece of circuitry. It is also acceptable to structure one processor by combining together a plurality of pieces of independent circuitry so as to realize the functions thereof. Furthermore, it is also acceptable to integrate two or more of the constituent elements illustrated in the drawings into one processor so as to realize the functions thereof.

The constituent elements of the apparatuses according to the above embodiments are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Further, it is possible to realize the medical information processing methods explained in the above embodiments, by causing a computer such as a personal computer or a workstation to execute a medical information processing program prepared in advance. The medical information processing program may be distributed via a network such as the Internet. Further, the medical information processing program may be executed, as being recorded on a non-transitory computer-readable recording medium such as a hard disk, a flexible disk (FD), a Compact Disk Read-Only Memory (CD-ROM), a Magneto Optical (MO) disk, a Digital Versatile Disk (DVD), or the like and being read by a computer from the recording medium.

According to at least one aspect of the embodiments described above, it is possible to assist using the information about the procedure performed on the patient P.

By using the X-ray diagnosis apparatus according to at least one aspect of the present embodiments, it is possible to assist using the information about the procedure performed on the examined subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Regarding the above embodiments, the following appendixes are disclosed as one aspect and optional feature of the invention.

### Appendix 1

A medical information processing apparatus comprising;
a first acquiring unit configured to acquire first information about a procedure performed on an examined subject so as to be kept in correspondence with time information; and
a second acquiring unit configured to acquire, on the basis of the first information, second information about the procedure so as to be kept in correspondence with time information.

### Appendix 2

The second acquiring unit may be configured to acquire the second information on the basis of a plurality of types of the first information.

### Appendix 3

The first information may be at least one selected from among: an optical camera image taken in a medical examination room where the procedure is performed; audio recorded in the medical examination room; a medical image taken of the examined subject; an imaging condition of the medical image; biological information data related to the examined subject acquired in the procedure; and information about a drug administered for the examined subject.

### Appendix 4

The optical camera image may be at least one selected from among: an image taken of a vicinity of hands of a first user; an image taken of a vicinity of hands of a second user different from the first user; an image taken of a face of the examined subject; an image taken of a face of the first user; and an image taken of a face of the second user.

### Appendix 5

The first acquiring unit may be configured to acquire, as the first information, an optical camera image taken in a medical examination room where the procedure is performed and a medical image taken of the examined subject, and

the second acquiring unit may be configured to acquire, as the second information, information indicating a medical tool used in the procedure on the basis of the optical camera image and the medical image.

### Appendix 6

The second acquiring unit may be configured to acquire, as the second information, information indicating that the medical tool has been replaced.

### Appendix 7

The second acquiring unit may be configured to acquire the information indicating the medical tool on the basis of registration information about medical tools that are usable in facility

### Appendix 8

The first acquiring unit may be configured to acquire, as the first information, an optical camera image taken in a medical examination room where the procedure is performed and information of the read identifier indicating a medical tool used in the procedure, and

the second acquiring unit may be configured to acquire, as the second information, information indicating a medical tool used in the procedure on the basis of the optical camera image and the information of the read identifier.

### Appendix 9

The first acquiring unit may be configured to acquire, as the first information, an optical camera image taken in a medical examination room where the procedure is performed, and

the second acquiring unit may be configured to acquire, as the second information, a clip image that is within the optical camera image and that includes a part rendering the medical tool used in the procedure.

### Appendix 10

The first acquiring unit may be configured to acquire, as the first information, one of an optical camera image rendering a medical tool used for administering a drug for the examined subject and audio recorded in a medical examination room where the procedure is performed, and

the second acquiring unit may be configured to acquire, as the second information, information indicating a type of the drug, on a basis of the one of the optical camera image and the audio.

### Appendix 11

A medical information processing apparatus comprising:
an acquiring unit configured to acquire information about a procedure performed on an examined subject so as to be kept in correspondence with time information; and
an output unit configured to output the information in accordance with the time information.

### Appendix 12

The output unit may be configured to output: first output information that is the information of a first type and is kept in correspondence with the time information indicating a first point in time; and second output information that is the information of a second type different from the first type and is kept in correspondence with the time information indicating the first point in time, and

at a time of outputting third output information that is the information of the first type and is kept in correspondence with the time information indicating a second point in time different from the first point in time, the output unit is configured to output fourth output information that is the information of the second type and is kept in correspondence with the time information indicating the second point in time.

### Appendix 13

The information may be medical images, and

as the information, the output unit may be configured to output: a first medical image kept in correspondence with the time information indicating a first point in time; and a second medical image that is a medical image kept in correspondence with the time information indicating a second point in time different from the first point in time and is acquired under an imaging condition similar to that of the first medical image.

### Appendix 14

The information may be at least one selected from among: an optical camera image taken in a medical examination room where the procedure is performed; audio recorded in the medical examination room; a medical image taken of the examined subject; an imaging condition of the medical image; biological information data related to the examined subject acquired in the procedure; and information about a drug administered for the examined subject.

### Appendix 15

The optical camera image may be at least one selected from among: an image taken of a vicinity of hands of a first user; an image taken of a vicinity of hands of a second user different from the first user; an image taken of a face of the examined subject; an image taken of a face of the first user; and an image taken of a face of the second user.

### Appendix 16

A medical information processing apparatus comprising:
an acquiring unit configured to acquire information about a procedure performed on an examined subject so as to be kept in correspondence with time information;
an analyzing unit configured to perform an analysis based on the information; and
an output unit configured to output a result of the analysis.

### Appendix 17

The acquiring unit may be configured to acquire, as the information, information indicating a type of a drug to be administered for the examined subject, and

on a basis of the information indicating the type of the drug and examined subject information, the analyzing unit may be configured to analyze suitability of the drug for the examined subject.

### Appendix 18

As the information, the acquiring unit may be configured to acquire: information indicating details of an instruction given by a first user performing the procedure to a second user performing the procedure; and an optical camera image related to the second user, and

the analyzing unit may be configured to analyze appropriateness of an action of the second user, on a basis of the information indicating the details of the instruction and the optical camera image.

### Appendix 19

The acquiring unit may be configured to acquire, as the information, an optical camera image related to the first user and the second user performing the procedure, and

on the basis of the optical camera image, the analyzing unit may be configured to analyze information that is related to the examined subject and is included only in a field of vision of one of the first and the second users.

### Appendix 20

The procedure may be intravascular intervention treatment performed by inserting a medical tool into a body of the examined subject,

the acquiring unit may be configured to acquire, as the information, a medical image taken of a range including the medical tool and an optical camera image related to a user operating the medical tool, and

the analyzing unit may be configured to analyze appropriateness of an operation performed on the medical tool by the user, on a basis of the medical image and the optical camera image.

### Appendix 21

The procedure may be treatment performed by inserting a first medical tool into a body of the examined subject,

the acquiring unit may be configured to acquire, as the information, a medical image taken of a range including the first medical tool, and

the analyzing unit may be configured to analyze an object different from the first medical tool appearing in the medical image, on a basis of the medical image and specification information of the first medical tool.

### Appendix 22

The output unit may be configured to update registration information of the object, on the basis of analysis result of the analyzing unit.

### Appendix 23

The procedure may be treatment performed by inserting a first medical tool into a body of the examined subject,

the acquiring unit may be configured to acquire, as the information, a medical image taken of a range including the first medical tool, and

the analyzing unit may be configured to: analyze a second medical tool different from the first medical tool appearing in the medical image, on a basis of the medical image and specification information of the first medical tool; and select a third medical tool to be used in the treatment on the basis of analysis result of the second medical tool.
vision of one of the first and the second users.

### Appendix 24

The procedure may be intravascular intervention treatment performed by inserting a medical tool into a body of the examined subject,

the acquiring unit may be configured to sequentially acquire, as the information, blood vessel fluoroscopic image in which the contrast of a blood vessel of the patient is enhanced, and

the analyzing unit may be configured to analyze blood flow state in the blood vessel fluoroscopic image in accordance with a purpose of the procedure.

### Appendix 25

The acquiring unit may be configured to acquire time-series information as the information, and

the analyzing unit may be configured to detect a fluctuation in the time-series information.

### Appendix 26

On a basis of the detected fluctuation and information about a procedure performed for the examined subject in relation to the procedure, the analyzing unit may be configured to judge whether or not the detected fluctuation is a fluctuation based on the procedure.

### Appendix 27

The acquiring unit may be configured to acquire the information related to the examined subject corresponding to a time after the procedure is started, and

the analyzing unit may be configured to estimate a state change that may occur in the examined subject, on a basis of the information.

### Appendix 28

The information may be at least one selected from among: an optical camera image taken in a medical examination room where the procedure is performed; audio recorded in the medical examination room; a medical image taken of the examined subject; an imaging condition of the medical image; biological information data related to the examined subject acquired in the procedure; and information about a drug administered for the examined subject.

### Appendix 29

The optical camera image may be at least one selected from among: an image taken of a vicinity of hands of a first user; an image taken of a vicinity of hands of a second user different from the first user; an image taken of a face of the examined subject; an image taken of a face of the first user; and an image taken of a face of the second user.

### Appendix 30

A medical information processing system comprising;
a first acquiring unit configured to acquire first information about a procedure performed on an examined subject so as to be kept in correspondence with time information; and
a second acquiring unit configured to acquire, on the basis of the first information, second information about the procedure so as to be kept in correspondence with time information.

### Appendix 31

A medical information processing system comprising:
an acquiring unit configured to acquire information about a procedure performed on an examined subject so as to be kept in correspondence with time information; and
an output unit configured to output the information in accordance with the time information.

### Appendix 32

A medical information processing system comprising:
an acquiring unit configured to acquire information about a procedure performed on an examined subject so as to be kept in correspondence with time information;
an analyzing unit configured to perform an analysis based on the information; and
an output unit configured to output a result of the analysis.

### Appendix 33

A medical information processing method comprising:
acquiring first information about a procedure performed on an examined subject so as to be kept in correspondence with time information; and
acquiring, on the basis of the first information, second information about the procedure so as to be kept in correspondence with time information.

### Appendix 34

A medical information processing method comprising:
acquiring information about a procedure performed on an examined subject so as to be kept in correspondence with time information; and
outputting the information in accordance with the time information.

### Appendix 35

A medical information processing method comprising:
acquiring information about a procedure performed on an examined subject so as to be kept in correspondence with time information;
performing an analysis based on the information; and
outputting a result of the analysis.

What is claimed is:

1. A medical information processing apparatus comprising processing circuitry configured:
to acquire first information about a procedure performed on an examined subject so as to be kept in correspondence with time information; and
to acquire, on a basis of the first information, second information about the procedure so as to be kept in correspondence with time information.

2. The medical information processing apparatus according to claim 1, wherein
as the first information, the processing circuitry is configured to acquire one of an optical camera image rendering a medical tool used for administering a drug for the examined subject and audio recorded in a medical examination room where the procedure is performed, and
as the second information, the processing circuitry is configured to acquire information indicating a type of the drug, on a basis of the one of the optical camera image and the audio.

3. A medical information processing apparatus comprising processing circuitry configured:
to acquire information about a procedure performed on an examined subject so as to be kept in correspondence with time information; and
to output the information in accordance with the time information.

4. The medical information processing apparatus according to claim 3, wherein
the processing circuitry is configured to output: first output information that is the information of a first type and is kept in correspondence with the time information indicating a first point in time; and second output information that is the information of a second type different from the first type and is kept in correspondence with the time information indicating the first point in time, and
at a time of outputting third output information that is the information of the first type and is kept in correspondence with the time information indicating a second point in time different from the first point in time, the processing circuitry is configured to output fourth output information that is the information of the second type and is kept in correspondence with the time information indicating the second point in time.

5. The medical information processing apparatus according to claim 3, wherein
the information is medical images, and
as the information, the processing circuitry is configured to output: a first medical image kept in correspondence with the time information indicating a first point in time; and a second medical image that is a medical image kept in correspondence with the time information indicating a second point in time different from the first point in time and is acquired under an imaging condition similar to that of the first medical image.

6. The medical information processing apparatus according to claim 3, wherein the information is at least one selected from among: an optical camera image taken in a medical examination room where the procedure is performed; audio recorded in the medical examination room; a medical image taken of the examined subject; an imaging condition of the medical image; biological information data related to the examined subject acquired in the procedure; and information about a drug administered for the examined subject.

7. The medical information processing apparatus according to claim 6, wherein the optical camera image is at least one selected from among: an image taken of a vicinity of hands of a first user; an image taken of a vicinity of hands of a second user different from the first user; an image taken of a face of the examined subject; an image taken of a face of the first user; and an image taken of a face of the second user.

8. A medical information processing apparatus comprising processing circuitry configured:
to acquire information about a procedure performed on an examined subject so as to be kept in correspondence with time information;
to perform an analysis based on the information; and
to output a result of the analysis.

9. The medical information processing apparatus according to claim 8, wherein
as the information, the processing circuitry is configured to acquire information indicating a type of a drug to be administered for the examined subject, and
on a basis of the information indicating the type of the drug and examined subject information, the processing circuitry is configured to analyze suitability of the drug for the examined subject.

10. The medical information processing apparatus according to claim 8, wherein
as the information, the processing circuitry is configured to acquire: information indicating details of an instruction given by a first user performing the procedure to a second user performing the procedure; and an optical camera image related to the second user, and
the processing circuitry is configured to analyze appropriateness of an action of the second user, on a basis of the information indicating the details of the instruction and the optical camera image.

11. The medical information processing apparatus according to claim 8, wherein
as the information, the processing circuitry is configured to acquire an optical camera image related to the first user and the second user performing the procedure, and
on the basis of the optical camera image, the processing circuitry is configured to analyze information that is related to the examined subject and is included only in a field of vision of one of the first and the second users.

12. The medical information processing apparatus according to claim 8, wherein
the procedure is intravascular intervention treatment performed by inserting a medical tool into a body of the examined subject,
as the information, the processing circuitry is configured to acquire a medical image taken of a range including the medical tool and an optical camera image related to a user operating the medical tool, and
the processing circuitry is configured to analyze appropriateness of an operation performed on the medical tool by the user, on a basis of the medical image and the optical camera image.

13. The medical information processing apparatus according to claim 8, wherein
the procedure is treatment performed by inserting a first medical tool into a body of the examined subject,
as the information, the processing circuitry is configured to acquire a medical image taken of a range including the first medical tool, and
the processing circuitry is configured to analyze a second medical tool being different from the first medical tool and appearing in the medical image, on a basis of the medical image and specification information of the first medical tool, and is configured to select a third medical tool to be used for the treatment in accordance with an analysis result of the second medical tool.

14. The medical information processing apparatus according to claim 8, wherein
the processing circuitry is configured to acquire time-series information as the information, and
the processing circuitry is configured to detect a fluctuation in the time-series information.

15. The medical information processing apparatus according to claim 14, wherein, on a basis of the detected fluctuation and information about a procedure performed for the examined subject in relation to the procedure, the processing circuitry is configured to judge whether or not the detected fluctuation is a fluctuation based on the procedure.

16. The medical information processing apparatus according to claim 8, wherein
the processing circuitry is configured to acquire the information related to the examined subject corresponding to a time after the procedure is started, and
the processing circuitry is configured to estimate a state change that may occur in the examined subject, on a basis of the information.

17. A medical information processing system comprising processing circuitry configured:
to acquire information about a procedure performed on an examined subject so as to be kept in correspondence with time information; and
to output the information in accordance with the time information.

18. A medical information processing system comprising processing circuitry configured:
to acquire information about a procedure performed on an examined subject so as to be kept in correspondence with time information;
to perform an analysis based on the information; and
to output a result of the analysis.

19. A medical information processing method comprising:
acquiring information about a procedure performed on an examined subject so as to be kept in correspondence with time information; and
outputting the information in accordance with the time information.

20. A medical information processing method comprising:
acquiring information about a procedure performed on an examined subject so as to be kept in correspondence with time information;
performing an analysis based on the information; and
outputting a result of the analysis.
